SEQUENCE LISTING (16 sequences)

SEQ ID NO: 1 - DNA, 49 bp, artificial
  Synthetic polyA
aataaaagatctttattttcattagatctgtgtgttggttttttgtgtg

SEQ ID NO: 2 - DNA, 766 bp, artificial
  synthetic polyA
attcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 3 - DNA, 507 bp, homo sapiens
ttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 4 - DNA, 446 bp, artificial
  modified intron 2/exon 3 sequence from human beta globin gene
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcctgttcatacctcttatcttcctcccacagctcctgggcaacgtgctggtctgtgtgctggcccatcactttggcaaagaattc

SEQ ID NO: 5 - DNA, 1188 bp, homo sapiens
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 6 - DNA, 1150 bp, homo sapiens
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctga

SEQ ID NO: 7 - DNA, 1188 bp, artificial
  hASAH1 co2
atgcctggcaggagctgtgtggccctggtgctgctggctgctgctgtgagctgtgctgtggcccagcatgcccccccctggacagaggactgcaggaagagcacctaccccccctctggccccacctacaggggggctgtgccctggtacaccatcaacctggacctgcccccctacaagaggtggcatgagctgatgctggacaaggcccctgtgctgaaggtgattgtgaacagcctgaagaacatgatcaacacctttgtgccctctggcaagatcatgcaggtggtggatgagaagctgcctggcctgctgggcaacttccctggcccctttgaggaggagatgaagggcattgctgctgtgacagacatccccctgggggagatcatcagcttcaacatcttctatgagctgttcaccatctgcacctccattgtggctgaggacaagaagggccacctgatccatggcaggaacatggactttggggtgttcctgggctggaacatcaacaatgacacctgggtgatcacagagcagctgaagcccctgacagtgaacctggacttccagaggaacaacaagacagtgttcaaggccagcagctttgctggctacgtgggcatgctgacaggcttcaagcctggcctgttcagcctgaccctgaatgagaggttcagcatcaatgggggctacctgggcatcctggagtggatcctgggcaagaaggatgtgatgtggattggcttcctgaccaggacagtgctggagaacagcaccagctatgaggaggccaagaacctgctgaccaagaccaagatcctggcccctgcctacttcatcctggggggcaaccagtctggggagggctgtgtgatcaccagggacaggaaggagagcctggatgtgtatgagctggatgccaagcagggcaggtggtatgtggtgcagaccaactatgacaggtggaagcaccccttcttcctggatgacaggaggacccctgccaagatgtgcctgaacaggaccagccaggagaacatcagctttgagaccatgtatgatgtgctgagcaccaagcctgtgctgaacaagctgacagtgtacaccaccctgattgatgtgaccaagggccagtttgagacctacctgagggactgccctgacccctgcattggctggtga

SEQ ID NO: 8 - DNA, 1188 bp, artificial
  hASAH1 co3
atgcctggcaggagctgtgtggccctggtgctgctggctgctgctgtgagctgtgctgtggcccagcacgcccccccctggacagaggactgcaggaagagcacctaccccccctctggccccacctacaggggggctgtgccctggtacacaatcaacctggacctgcccccctacaagaggtggcacgagctgatgctggacaaggcccctgtgctgaaggtgattgtgaacagcctgaagaatatgatcaacacctttgtgccctctggcaagattatgcaggtggtggacgagaagctgcctggcctgctgggcaacttccctggcccctttgaggaggagatgaaggggattgctgctgtgacagatatccccctgggggagattatcagcttcaatatcttctacgagctgttcacaatctgcacctctattgtggctgaggacaagaagggccacctgatccacggcaggaatatggactttggggtgttcctgggctggaatatcaacaacgacacctgggtgatcacagagcagctgaagcccctgacagtgaacctggacttccagaggaacaacaagacagtgttcaaggccagcagctttgctggctacgtggggatgctgacaggcttcaagcctggcctgttcagcctgaccctgaacgagaggttctctatcaacgggggctacctggggatcctggagtggatcctgggcaagaaggacgtgatgtggattggcttcctgaccaggacagtgctggagaacagcaccagctacgaggaggccaagaacctgctgaccaagaccaagatcctggcccctgcctactttatcctggggggcaaccagtctggggagggctgtgtgatcaccagggacaggaaggagagcctggacgtgtacgagctggacgccaagcagggcaggtggtacgtggtgcagaccaactacgacaggtggaagcaccccttcttcctggacgacaggaggacccctgccaagatgtgcctgaacaggaccagccaggagaatatcagctttgagacaatgtacgacgtgctgagcaccaagcctgtgctgaacaagctgacagtgtacaccaccctgattgacgtgaccaagggccagtttgagacctacctgagggactgccctgacccctgtattggctggtag

SEQ ID NO: 9 - DNA, 1708 bp, artificial
  CAG promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaactgggcggggagggccttcgtgcgtcgccgcgccgccgtccccttctccatctccagcctcggggctgccgcagggggacggctgccttcgggggggacggggcagggcggggttcggcttctggcgtgtgaccggcggcctctgctaaccatgttcatgccttcttctttttcctacagctcctgggcaacgtgctggttattgtgctgtctcatcattttggcaaagaatt

SEQ ID NO: 10 - DNA, 678 bp, artificial
  WPRE4
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctgga

SEQ ID NO: 11 - DNA, 25 bp, artificial
  primer
cagaaggtatgcggcatcgtcatac

SEQ ID NO: 12 - DNA, 26 bp, artificial
  primer
agggccatacagagaaaccctgtctc

SEQ ID NO: 13 - DNA, 22 bp, artificial
  primer
tcaaggcttgactttggggcac

SEQ ID NO: 14 - DNA, 26 bp, artificial
  primer
gctggacgtaaactcctcttcagacc

SEQ ID NO: 15 - DNA, 145 bp, artificial
  AAV2 ITR 5′
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 16 - DNA, 145 bp, artificial
  AAV2 ITR 3′
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2023/0151390 A1
(43) Pub. Date: May 18, 2023

(54) VECTORS FOR THE TREATMENT OF ACID CERAMIDASE DEFICIENCY

(71) Applicants: GENETHON, EVRY (FR); INSERM (INSTITUT NATIONAL DE LA SANTÉ ET DE LA RECHERCHE MÉDICALE), PARIS (FR); UNIVERSITE D'EVRY VAL D'ESSONNE, EVRY (FR)

(72) Inventors: ANA BUJ BELLO, PARIS (FR); JÉRÔME DENARD, ST PIERRE DU PERRAY (FR); MARTINA MARINELLO, ANTONY (FR)

(21) Appl. No.: 17/996,085

(22) PCT Filed: Apr. 14, 2021
(86) PCT No.: PCT/EP2021/059709

§ 371 Date: Oct. 13, 2022

(30) Foreign Application Priority Data
Apr. 14, 2020 (WO) 20169463.5

(51) Int. Cl. C12N 15/86; A61P 3/00; A61K 38/50

(52) U.S. Cl. CPC C12N 15/86; A61P 3/00; A61K 38/50; C12Y 305/01023; C12N 2750/14143; C12N 2750/14171; C12N 2800/22; C12N 2830/42; C12N 2830/50; A61K 48/00

(57) ABSTRACT

The present invention relates to a vector comprising an ASAH1 open reading frame, for use in the treatment of acid ceramidase deficiency.

### FIELD OF THE INVENTION

The present invention relates to a vector comprising an ASAH1 open reading frame, for use in the treatment of acid ceramidase deficiency, such as spinal muscular atrophy associated with progressive myoclonic epilepsy or Farber disease.

### BACKGROUND OF THE INVENTION

Acid ceramidase deficiency is a spectrum of disorders that includes a rare lysosomal storage disorder called Farber disease (FD) and a rare epileptic disorder called spinal muscular atrophy with progressive myoclonic epilepsy (SMA-PME). Both FD and SMA-PME are caused by mutations in the acid ceramidase (ASAH1) gene, which lead to decreased acid ceramidase activity and in turn to ceramide accumulation and various pathological manifestations. FD is an early onset disease resulting from an acid ceramidase activity below 10%, whereas a higher residual activity is responsible for SMA-PME, which has a later onset phenotype restricted to the CNS and starting with lower motor neuron disease. FD starts with subcutaneous lipogranulomata, joint pains and hoarseness of the voice. Neurological and visceral involvement are also observed that lead to psychomotor retardation and hepatosplenomegaly, respectively. Most FD patients show growth and developmental impairment and do not live past 2 years of age. Nodules, composed of foamy histiocytes and macrophages, and inflammation are typically present in affected tissues, including the bone marrow, highlighting the potential role of the hematopoietic system in the disease. In addition, leukocytes appear to be primarily dysregulated in Farber disease. SMA-PME appears clinically during infancy as a lower motor neuron disease with muscle weakness, difficulty in walking and tremors. Epilepsy with myoclonic seizures usually develop during late childhood, and other clinical manifestations such as cognitive decline, scoliosis and hearing loss may also appear with disease evolution. The death of patients generally occurs during the teenage years due to respiratory failure.

There is currently no cure for acid ceramidase deficiency (Yu et al., 2018; Orphanet Journal or Rare Diseases; 13: 121). Therefore, current treatment strategies are limited to symptom management and supportive care. Hematopoietic stem cell transplantation (HSCT) showed some promising results with a substantial improvement of mobility and pain in a number of FD patients lacking CNS involvement. However, HSCT performed in patients with classical FD with CNS complications did not reverse the neurological phenotypes and the patients deteriorated over time. Ex vivo gene therapy was also proposed, wherein hematopoietic stem cells (HSCs) are isolated from a patient or donor, modified by genetic correction using a lentiviral vector, and then transplanted to the patient in need thereof. However, this approach may not be efficacious in resolving the neurological phenotypes of ASAH1 deficiency, as no significant changes were observed in the brain of non-human primates receiving lentivector/acid ceramidase-transduced hematopoietic cells (Walia et al 2011; Hum Gene Ther; 22, p. 679-687). Enzyme replacement therapy (ERT) is also under development, involving the injection of recombinant acid ceramidase to a patient in need thereof. However, ERT strategies face limitations with respect to neurological phenotypes, because of the poor ability for an enzyme to cross the blood-brain barrier.

Therefore, there is still an urgent need of an efficient therapy against acid ceramidase deficiency, such as SMA-PME and Farber disease.

### SUMMARY OF THE INVENTION

The present invention stems from the surprising finding that adeno-associated (AAV) vectors comprising a ASAH1 open reading frame (ORF) are efficient gene therapy vectors for the treatment of FD and SMA-PME. In particular, it is herein shown that thanks to the invention, both CNS and peripheral alterations are corrected. The present invention is a major improvement over the currently developed therapies because to date, no other treatment is able to correct both alterations.

In a first aspect, the invention relates to a recombinant AAV (rAAV) vector comprising a ASAH1 ORF. This vector can be used in a method for the treatment of an acid ceramidase deficiency. In particular, the rAAV vector can be used in a method for the treatment of a disorder selected from Farber disease (FD) and spinal muscular atrophy associated with progressive myoclonic epilepsy (SMA-PME). In a particular embodiment, the AAV vector is for use in a method for the treatment of cystic fibrosis. In a particular embodiment, the vector is an AAV vector able to cross the blood-brain barrier. Illustrative AAV vectors having the ability to cross the blood-brain barrier include, without limitation, vectors comprising an AAV9 capsid, an AAV9P1 capsid, an AAVpo1A1 capsid, an AAV10 capsid, such as an AAVrh.10 or AAVcy.10 capsid, a AAVrh.39 capsid or a clade F AAVHSC capsid. In a further embodiment, the AAV vector comprises an AAV9 capsid, an AAV10 capsid, such as an AAVrh.10 or AAVcy.10 capsid, a AAVrh.39 capsid or a clade F AAVHSC capsid. In another embodiment, the AAV vector comprises an AAV9 or AAV10 capsid. In yet another embodiment, the AAV vector comprises an AAV9 capsid.

For expression, the ASAH1 ORF is comprised in an expression cassette, operatively linked to regulatory sequences. Such regulatory sequences include, without limitation, promoters, introns, enhancers and polyadenylation signals. In a particular embodiment, the ASAH1 ORF is under the control of an ubiquitous promoter. In another embodiment, the ubiquitous promoter is selected from a CAG promoter, a PGK promoter or a beta-glucuronidase promoter. In a further embodiment, the ASAH1 ORF is under the control of a CAG promoter or a PGK promoter. In a particular embodiment, the promoter is the CAG promoter. In a particular embodiment, the CAG promoter has the sequence shown in SEQ ID NO:9, or said promoter is a functional variant of said promoter having a nucleotide sequence that is at least 80% identical to SEQ ID NO:9, in particular at least 85%, at least 90%, at least 95% or at least 99% identical to SEQ ID NO:9. In a particular embodiment, the promoter consists of a nucleic acid sequence at least 99% identical to SEQ ID NO:9 or consists of the nucleic acid sequence shown in SEQ ID NO:9. In a particular embodiment, the PGK promoter has the sequence shown in SEQ ID NO:3, or said promoter is a functional variant of said promoter having a nucleotide sequence that is at least 80% identical to SEQ ID NO:3, in particular at least 85%, at least 90%, at least 95% or at least 99% identical to SEQ ID NO:3. In a particular embodiment, the promoter consists of a nucleic acid sequence at least 99% identical to SEQ ID NO:3 or consists of the nucleic acid sequence shown in SEQ ID NO:3.

In another embodiment, depending on the promoter included into the expression cassette, said cassette may further comprise an intron. For example, the cassette may further comprise a modified intron 2/exon 3 sequence from the human β globin gene. In a particular embodiment, if the promoter is a PGK promoter, the cassette may further comprise an intron, such as a modified intron 2/exon 3 sequence from the human β globin gene. In a particular embodiment, the modified intron 2/exon 3 sequence from the human β globin gene has the sequence shown in SEQ ID NO:4, or is a functional variant of the sequence shown in SEQ ID NO:4, which has at least 80% identity with SEQ ID NO:4, in particular at least 85%, at least 90%, at least 95% or at least 99% identity with SEQ ID NO:4. In a particular embodiment, the intron comprised into the cassette consists of a nucleic acid sequence at least 99% identical to SEQ ID NO:4 or consists of the nucleic acid sequence shown in SEQ ID NO:4.

In yet another embodiment, the expression cassette comprises a polyadenylation signal selected in the group consisting of the polyadenylation signal of the ASAH1 gene, a polyadenylation signal from the human β globin gene (HBB pA), the bovine growth hormone polyadenylation signal, the SV40 polyadenylation signal, and a synthetic polyA, such as the synthetic polyadenylation signal of SEQ ID NO:1. In a particular embodiment, the polyadenylation signal is a HBB polyadenylation signal, such as a HBB polyadenylation signal having a sequence consisting of SEQ ID NO:2, or a functional variant thereof having a nucleotide sequence that is at least 80% identical to the sequence shown in SEQ ID NO:2, in particular at least 85%, at least 90%, at least 95% or at least 99% identical to SEQ ID NO:2.

In a particular embodiment, the ASAH1 ORF is a human ASAH1 ORF deriving from an ASAH1 wild-type gene. In a particular embodiment, the nucleic acid sequence of the ASAH1 ORF is a wild-type sequence or an optimized sequence.

The expression cassette introduced in the genome is flanked by sequences suitable for the packaging of said expression cassette into an AAV viral vector, referred to as AAV inverted terminal repeat (ITR) sequences. Therefore, the expression cassette is flanked by an AAV 5′-ITR and an AAV 3′-ITR for its further packaging into an AAV vector. In a particular embodiment, the expression cassette is flanked by an AAV2 5′-ITR and an AAV2 3′-ITR.

The genome of the rAAV vector may be a single-stranded genome, or a self-complementary genome. In a particular embodiment, the genome of the AAV vector is single-stranded.

In a particular embodiment, the rAAV vector of the invention comprises an expression cassette comprising, in this order:
a CAG promoter;
an ASAH1 ORF; and
a polyadenylation signal sequence, such as a HBB polyadenylation signal.
   In a particular embodiment, the CAG promoter has the sequence shown in SEQ ID NO:9, or said promoter is a functional variant of said promoter having a nucleotide sequence that is at least 80% identical to SEQ ID NO:9, in particular at least 85%, at least 90%, at least 95% or at least 99% identical to SEQ ID NO:9. In a particular embodiment, the promoter consists of a nucleic acid sequence at least 99% identical to SEQ ID NO:9 or consists of the nucleic acid sequence shown in SEQ ID NO:9.

In yet another embodiment, the rAAV vector of the invention comprises an expression cassette comprising, in this order:
a PGK promoter;
a modified intron 2/exon 3 sequence from the human β globin gene;
an ASAH1 ORF; and
a polyadenylation signal, such as a HBB polyadenylation signal.
   In a particular embodiment, the PGK promoter has the sequence shown in SEQ ID NO:3, or said promoter is a functional variant of said promoter having a nucleotide sequence that is at least 80% identical to SEQ ID NO:3, in particular at least 85%, at least 90%, at least 95% or at least 99% identical to SEQ ID NO:3. In a particular embodiment, the promoter consists of a nucleic acid sequence at least 99% identical to SEQ ID NO:3 or consists of the nucleic acid sequence shown in SEQ ID NO:3.

In a particular embodiment, the modified intron 2/exon 3 sequence from the human β globin gene has the sequence shown in SEQ ID NO:4, or is a functional variant of the sequence shown in SEQ ID NO:4, which has at least 80% identity with SEQ ID NO:4, in particular at least 85%, at least 90%, at least 95% or at least 99% identity with SEQ ID NO:4. In a particular embodiment, the intron comprised into the cassette consists of a nucleic acid sequence at least 99% identical to SEQ ID NO:4 or consists of the nucleic acid sequence shown in SEQ ID NO:4.

In a second aspect, the invention relates to the rAAV vector of the invention, for use in a method for the treatment of a disease by gene therapy. In a particular embodiment, the invention relates to the rAAV vector of the invention, for use in a method for the treatment of acid ceramidase deficiency. In a particular embodiment, the vector is for use in a method for the treatment of cystic fibrosis. In a preferred embodiment, the acid ceramidase deficiency is FD or SMA-PME. In a particular embodiment, the acid ceramidase deficiency is SMA-PME. In a particular embodiment, the rAAV vector of the invention is for use in a method for the treatment of FD. In yet another embodiment, the rAAV vector is for use in a method for the treatment of at least one neurological and/or peripheral clinical manifestations of Farber disease (FD). In a further embodiment, the rAAV vector is for use in a method for treating at least hematological manifestations of FD. In an embodiment, the rAAV vector is for use in a method for treating neurological and hematological manifestations of FD.

In a particular embodiment, the rAAV vector as defined throughout this application is a rAAV9 vector, for use in a method for the treatment of a disease by gene therapy. In a particular embodiment, the invention relates to the rAAV9 disclosed herein, for use in a method for the treatment of acid ceramidase deficiency. In a particular embodiment, the vector is for use in a method for the treatment of cystic fibrosis. In a preferred embodiment, the acid ceramidase deficiency is FD or SMA-PME. In a particular embodiment, the rAAV9 vector is for use in a method for the treatment of FD. In yet another embodiment, the rAAV9 vector is for use in a method for the treatment of at least one neurological and/or peripheral clinical manifestations of Farber disease (FD). In a further embodiment, the rAAV vector is for use in a method for treating at least hematological manifestations of FD. In an embodiment, the rAAV9 vector is for use in a method for treating neurological and hematological manifestations of FD.

In another embodiment, said rAAV vector is for administration into the cerebrospinal fluid of a subject, in particular by intrathecal and/or intracerebroventricular injection. Alternatively, said rAAV vector is for peripheral administration, such as for intravascular (e.g. intravenous or intra-arterial), intramuscular or intraperitoneal administration.

In yet another embodiment, the rAAV vector comprises an AAV9 capsid, the ASAH1 ORF is under the control of a CAG promoter, and the rAAV vector is for intravascular administration. In an embodiment, the rAAV vector comprises an AAV9 capsid, the ASAH1 ORF is under the control of a CAG promoter, and the rAAV vector is for intravenous administration. In an embodiment, the rAAV vector comprises an AAV9 capsid, the ASAH1 ORF is under the control of a CAG promoter, and the rAAV vector is for intra-arterial administration.

In yet another embodiment, the rAAV vector comprises an AAV9 capsid, the ASAH1 ORF is under the control of a PGK promoter, and the rAAV vector is for intravascular administration. In an embodiment, the rAAV vector comprises an AAV9 capsid, the ASAH1 ORF is under the control of a PGK promoter, and the rAAV vector is for intravenous administration. In an embodiment, the rAAV vector comprises an AAV9 capsid, the ASAH1 ORF is under the control of a PGK promoter, and the rAAV vector is for intra-arterial administration.

### BRIEF DESCRIPTION OF THE DRAWINGS

FIG. 1: Kaplan-Meyer survival curve of untreated Asah^{P361R/P361R} mice, wild-type animals and Asah^{P361R/P361R} mice treated intravenously at 3 weeks of life with a rAAV vector comprising the hASAH1 transgene under the control of the CAG promoter at 5×10¹³ vg/kg.

FIG. 2: Kaplan-Meyer survival curve of untreated Asah^{P361R/P361R} mice, wild-type animals and Asah^{P361R/P361R} mice treated intravenously at 7 weeks of life with a rAAV vector comprising the hASAH1 transgene under the control of the CAG promoter at 5×10¹³ vg/kg.

FIG. 3: body weight assessment of untreated Asah^{P361R/P361R} mice, wild-type animals and Asah^{P361R/P361R} mice treated intravenously at 3 weeks of life with a rAAV vector comprising the hASAH1 transgene under the CAG promoter at 5×10¹³ vg/kg. All groups were sex balanced. Data are expressed as the mean±SEM, and differences between the curves of the WT+PBS, Asah^{P361R/P361R}+PBS and Asah^{P361R/P361R}+AAV-hASAH1 were compared by Multiple T-test (* P<0,05; ** P<0,0001).

FIG. 4: body weight assessment of untreated Asah^{P361R/P361R} mice, wild-type animals and Asah^{P361R/P361R} mice treated intravenously at 7 weeks of life with a rAAV vector comprising the hASAH1 transgene under the CAG promoter at 5×10¹³ vg/kg. All groups were sex balanced. Data are expressed as the mean±SEM, and differences between the curves of the WT+PBS, Asah^{P361R/P361R}+PBS and Asah^{P361R/P361R}+AAV-hASAH1 were compared by Multiple T-test (* P<0,05; ** P<0.001; $ P<0,0001).

FIG. 5: ratio of tissue weight over total body weight (mg/g) in mice injected at 3 weeks of age after 10 weeks of treatment. Results are expressed as the mean±SEM. The differences between groups were analyzed by Multiple T-test (*P<0.005).

FIG. 6: ratio of tissue weight over total body weight (mg/g) in mice injected at 7 weeks of age after 6 months of treatment. Results are expressed as the mean±SEM. The differences between groups were analyzed by Multiple T-test (No statistically significant differences).

FIG. 7: assessment of the effect of a rAAV vector comprising the hASAH1 transgene on complete blood cell count after 4 weeks of treatment. Mice treated at 3 weeks of age. Histograms showing results in untreated Asah^{P361R/P361R} mice, wild-type animals and Asah^{P361R/P361R} mice treated intravenously with a rAAV vector comprising the hASAH1 transgene under the CAG promoter at 5×10¹³ vg/kg. Results are expressed as the mean±SEM. The differences between groups were analyzed by Multiple T-test (*P<0.001).

FIG. 8: assessment of the effect of a rAAV vector comprising the hASAH1 transgene on complete blood cell count after 4 weeks of treatment. Mice treated at 7 weeks of age. Histograms showing results in untreated Asah^{P361R/P361R} mice, wild-type animals and Asah^{P361R/P361R} mice treated intravenously with a rAAV vector comprising the hASAH1 transgene under the CAG promoter at 5×10¹³ vg/kg. Results are expressed as the mean±SEM. The differences between groups were analyzed by Multiple T-test (* P<0,05; ** P<0.001).

FIG. 9: muscle strength assessment of untreated Asah^{P361R/P361R} mice, wild-type animals and Asah^{P361R/P361R} mice treated via the intravenous route with a rAAV vector comprising the hASAH1 transgene at 3 weeks of age. Results are expressed as the mean±SEM. The differences between groups were analyzed by Multiple T-test (* P<0,01; ** P<0,0001).

FIG. 10: muscle strength assessment of untreated Asah^{P361R/P361R} mice, wild-type animals and Asah^{P361R/P361R} mice treated via the intravenous route with a rAAV vector comprising the hASAH1 transgene. Mice treated at 7 weeks of age. Results are expressed as the mean±SEM. The differences between groups were analyzed by Multiple T-test (* P<0,05; ** P<0,0005).

FIG. 11: body weight assessment of untreated Asah^{P361R/P361R} mice, wild-type animals and Asah^{P361R/P361R} mice treated with an intracerebroventricular injection at birth (P0) of a rAAV vector comprising the hASAH1 transgene under the CAG promoter at 1×10¹³ vg/kg. Results are expressed as the mean±SEM. The differences between groups were analyzed by Multiple T-test (* P<0,05; ** P<0,00001).

FIG. 12: muscle strength assessment of untreated Asah^{P361R/P361R} mice, wild-type animals and Asah^{P361R/P361R} mice treated with an intracerebroventricular injection at birth (P0) of a rAAV vector comprising the hASAH1 transgene under the CAG promoter at 1×10¹³ vg/kg. Results are expressed as the mean±SEM. The differences between groups were analyzed by Multiple T-test (*P<0,005).

### DETAILED DESCRIPTION OF THE INVENTION

According to the present invention, the term “about”, when referring to a numerical value, means plus or minus 5% of this numerical value.

In the context of the present invention, a vector qualified as a “AAVx vector” or rAAVx” vector is a vector comprising a serotype x capsid. For example, a vector comprising an AAV9 capsid or an AAV10 capsid are referred to as “AAV9 vector” or “AAVrh10 vector”, respectively, independently of the serotype the genome contained in the rAAV vector is derived from. Accordingly, an AAV9 vector may be a vector comprising an AAV9 capsid and an AAV9 derived genome (i.e. comprising AAV9 ITRs) or a pseudotyped vector comprising an AAV9 capsid and a genome derived from a serotype different from the AAV9 serotype. Likewise, an AAVrh.10 vector may be a vector comprising an AAVrh.10 capsid and an AAVrh.10 derived genome (i.e. comprising AAVrh10 ITRs) or a pseudotyped vector comprising an AAVrh.10 capsid and a genome derived from a serotype different from the AAVrh.10 serotype. This definition applies mutatis mutandis to other AAV vector comprising a capsid different from the AAV9 and AAV10 capsids.

### Expression Cassette

The rAAV vector of the invention is for the delivery of an ASAH1 ORF. Said gene is comprised into an expression cassette comprising suitable regulatory elements.

In a particular embodiment, the ASAH1 ORF is from a human ASAH1 (hASAH1) gene. Any functional ASAH1 gene may be used. In a particular embodiment, the nucleic acid sequence of the hASAH1 ORF is derived from the Homo sapiens N-acylsphingosine amidohydrolase 1 (ASAH1), transcript variant 1, mRNA sequence having the Genbank accession No. NM_177924, as shown in SEQ ID NO:5 or the Homo sapiens N-acylsphingosine amidohydrolase 1 (ASAH1), transcript variant 2, mRNA sequence having the Genbank accession No. NM_004315.5, as shown in SEQ ID NO:6, in particular from transcript variant 1 shown in SEQ ID NO:5. In a particular embodiment, nucleic acid sequence of the hASAH1 ORF used in the present invention consists of or comprises the sequence shown in SEQ ID NO:5 or SEQ ID NO:6, in particular the sequence shown in SEQ ID NO:5.

In another particular embodiment, the nucleic acid sequence of the ASAH1 ORF is optimized. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimised sequence. In a particular embodiment of the invention, such sequence optimized nucleotide sequence is codon-optimized to improve its expression in human cells compared to non-codon optimized nucleotide sequences coding for the same protein, for example by taking advantage of the human specific codon usage bias.

In a particular embodiment, the optimized coding sequence is codon optimized, and/or has an increased GC content and/or has a decreased number of alternative open reading frames, and/or has a decreased number of splice donor and/or splice acceptor sites, as compared to the wild-type coding sequence (such as the wild-type human ASAH1 sequence of SEQ ID NO:5 or 6, in particular of SEQ ID NO:5).

In a particular embodiment, the nucleic acid sequence of the ASAH1 is at least 70% identical, in particular at least 75% identical, at least 80% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the sequence shown in SEQ ID NO:5 or 6, in particular in SEQ ID NO:5.

As mentioned above, in addition to the GC content and/or number of ARFs, sequence optimization may also comprise a decrease in the number of CpG islands in the sequence and/or a decrease in the number of splice donor and acceptor sites. Of course, as is well known to those skilled in the art, sequence optimization is a balance between all these parameters, meaning that a sequence may be considered optimized if at least one of the above parameters is improved while one or more of the other parameters is not, as long as the optimized sequence leads to an improvement of the transgene, such as an improved expression and/or a decreased immune response to the transgene in vivo.

In addition, the adaptiveness of a nucleotide sequence of the ASAH1 ORF to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al, Gene. 1997, 199:293-301; zur Megede et al, Journal of Virology, 2000, 74: 2628-2635).

In a particular embodiment, the nucleic acid sequence of the ASAH1 ORF consists of or comprises a wild-type sequence such as the sequence shown in SEQ ID NO:5 or SEQ ID NO:6, in particular SEQ ID NO:5, or an optimized sequence, such as the sequence shown in SEQ ID NO:7 or SEQ ID NO:8. In a further particular embodiment, the nucleic acid sequence of the ASAH1 ORF consists of or comprises a wild-type sequence such as the sequence shown in SEQ ID NO:5 or SEQ ID NO:6, in particular SEQ ID NO:5.

The genome of the rAAV vector comprises an expression cassette including the ASAH1 ORF. In the context of the present invention, an “expression cassette” is a nucleic acid sequence comprising a gene (here, the ASAH1 ORF) operably linked to sequences allowing the expression of said gene in an eukaryotic cell. In the rAAV vectors of the present invention, the ASAH1 ORF may be operably linked to one or more expression control sequences and/or other sequences improving the expression of the gene. As used herein, the term “operably linked” refers to a linkage of polynucleotide elements in a functional relationship. A nucleic acid is “operably linked” when it is placed into a functional relationship with another nucleic acid sequence. For instance, a promoter, or another transcription regulatory sequence, is operably linked to a coding sequence if it affects the transcription of the coding sequence. Such expression control sequences are known in the art, such as promoters, enhancers, introns and polyadenylation signals.

In the rAAV vector of the invention, the ASAH1 ORF is operably linked to a promoter.

According to a particular embodiment, an ubiquitous promoter is used. Representative ubiquitous promoters include, without limitation:
the CAG promoter, which includes the cytomegalovirus enhancer/chicken beta actin promoter, the first exon and the first intron of the chicken beta-actin gene and the splice acceptor of the rabbit beta-globin gene (SEQ ID NO:9);
a phosphoglycerate kinase 1 (PGK) promoter, such as the human PGK promoter shown in SEQ ID NO:3;
the cytomegalovirus enhancer/promoter (CMV) (optionally with the CMV enhancer) (see, e.g., Boshart et al, Cell, 41:521-530 (1985));
the SV40 early promoter;
the retroviral Rous sarcoma virus (RSV) LTR promoter (optionally with the RSV enhancer);
the dihydrofolate reductase promoter;
the β-actin promoter; and
the EF1 promoter.
   Other ubiquitous promoters useful in the practice of the present invention include, without limitation, the CB7 promoter, the beta-glucosidase (GUSB) promoter and the JeTi promoter.

According to another embodiment, in particular if the rAAV vector is for use in a method for the treatment of SMA-PME, the promoter may be a promoter driving selective expression into neurons or glial cells. Representative promoters driving expression into neurons include, without limitation, the promoter of the Calcitonin Gene-Related Peptide (CGRP), a known motor neuron-derived factor. Other neuron-selective promoters include the promoters of Choline Acetyl Transferase (ChAT), Neuron Specific Enolase (NSE), Synapsin, Hb9 and ubiquitous promoters including Neuron-Restrictive Silencer Elements (NRSE). Representative promoters driving selective expression in glial cells include the promoter of the Glial Fibrillary Acidic Protein gene (GFAP).

According to another embodiment, if the rAAV vector is for use in a method for the treatment of cystic fibrosis, the promoter is a promoter driving expression of the ASAH1 protein in lung epithelial cells. Representative promoters suitable to drive expression in lung epithelial cells include, without limitation, ubiquitous promoters and promoters driving selective expression in epithelial cells of internal organs, in particular selective expression in lung epithelial cells. Representative promoters useful in the practice of the invention for the treatment of cystic fibrosis include, without limitation, the human surfactant protein B (SP-B) promoter (described in Strayer et al., 2002, Am J Physiol Lung Cell Mol Physiol, 282(3): L394-404), the human cytokeratin 18 (K18) promoter and constructs comprising the same (such as those described in Chow et al., 1997, PNAS, 94(26), p. 14695), the surfactant protein C (SP-C) promoter (described in Zhuo et al., 2006, Transgenic Research, 15, p. 543) and the CTP:phosphocholine cytidylyltransferase promoter (CCTa) (described in Zhou et al., 2004, Am J Respir Cell Mol Biol 30(1), p. 61). In a particular embodiment, the rAAV vector for use in the treatment of cystic fibrosis is an ubiquitous promoter, such as a CAG or PGK promoter as disclosed herein.

In a particular embodiment, the promoter is selected from a CAG promoter or a PGK promoter.

In a further particular embodiment, the CAG promoter consists of the sequence shown in SEQ ID NO:9, or is a functional variant of the sequence shown in SEQ ID NO:9, having a nucleotide sequence that is at least 80% identical to the sequence shown in SEQ ID NO:9, in particular at least 85%, at least 90%, at least 95% or at least 99% identical to SEQ ID NO:9. In a particular embodiment, the promoter consists of a nucleic acid sequence which is a functional variant of the CAG promoter, which has at least 99% identical to SEQ ID NO:9. In yet another embodiment, the promoter is the CAG promoter having a nucleic acid sequence consisting of SEQ ID NO:9.

In a further particular embodiment, the PGK promoter consists of the sequence shown in SEQ ID NO:3, or is a functional variant of the sequence shown in SEQ ID NO:3, having a nucleotide sequence that is at least 80% identical to the sequence shown in SEQ ID NO:3, in particular at least 85%, at least 90%, at least 95% or at least 96%, 97%, 98% or 99% identical to SEQ ID NO:3. In a particular embodiment, the promoter consists of a nucleic acid sequence which is a functional variant of the PGK promoter, which has at least 99% identical to SEQ ID NO:3. In yet another embodiment, the promoter is the PGK promoter having a nucleic acid sequence consisting of SEQ ID NO:3.

In the context of the present invention, a functional variant of a promoter is a sequence deriving therefrom by one or more nucleotide modifications, such as nucleotide substitution, addition or deletion, that results in the same or substantially the same level of expression (e.g. ±20%, such as ±10%, ±5% or ±1%) of the ASAH1 ORF operatively linked thereto.

Other regulatory elements may be located downstream of the promoter. For example, one can cite the provision of an intron to increase expression from the expression cassette. Incorporation of such further regulatory elements will depend on the promoter used in the expression cassette and the resulting size of the cassette. In a particular embodiment, the expression cassette may comprise a further regulatory element downstream of the promoter, such as a sequence composed of a modified intron 2/exon 3 sequence from the human β globin gene. In a particular embodiment, the modified intron 2/exon 3 sequence from the human β globin gene has the sequence shown in SEQ ID NO:4, or is a functional variant of the sequence shown in SEQ ID NO:4, which has at least 80% identity with SEQ ID NO:4, in particular at least 85%, at least 90%, at least 95% or at least 96%, 97%, 98% or 99% identity with SEQ ID NO:4. In a particular embodiment, the further regulatory element is a functional variant of the modified intron 2/exon 3 sequence from the human β globin gene, which consists of a nucleic acid sequence having at least 99% identical to SEQ ID NO:4. In yet another embodiment, the further regulatory element is the modified intron 2/exon 3 sequence consisting of the sequence shown in SEQ ID NO:4. In the context of the present invention, a functional variant of the modified intron 2/exon 3 sequence from the human β globin gene is a sequence deriving therefrom by one or more nucleotide modifications, such as nucleotide substitution, addition or deletion, that results in the same or substantially the same level of expression (e.g. ±20%, such as ±10%, ±5% or ±1%) of the ASAH1 ORF operatively linked thereto.

In a particular embodiment of the invention, the expression cassette comprises a PGK promoter, such as the promoter having the sequence shown in SEQ ID NO:3 and, located downstream said promoter and upstream the ASAH1 ORF, the sequence composed of a modified intron 2/exon 3 sequence from the human β globin gene as shown in SEQ ID NO:4.

The polyadenylation signal in the expression cassette of the invention may be derived from a number of genes. Illustrative polyadenylation signals include, without limitation, the ASAH1 gene polyadenylation signal, the human β globin gene (HBB) polyadenylation signal, the bovine growth hormone polyadenylation signal and the SV40 polyadenylation signal. In a particular embodiment, the polyadenylation signal is a HBB polyadenylation signal, such as a HBB polyadenylation signal having a sequence consisting of the sequence shown in SEQ ID NO:2. In a particular embodiment, the HBB polyadenylation signal is a functional variant of the sequence shown in SEQ ID NO:2, which has at least 80% identity with SEQ ID NO:2, in particular at least 85%, at least 90%, at least 95% or at least 96%, 97%, 98% or 99% identity with SEQ ID NO:2. In another particular embodiment, the polyadenylation signal is a functional variant of the HBB polyadenylation signal which has a sequence at least 99% identical to the sequence shown in SEQ ID NO:2. In a further embodiment, the polyadenylation signal is the HBB polyadenylation signal consisting of the sequence shown in SEQ ID NO:2. In the context of the present invention, a functional variant of the HBB polyadenylation signal is a sequence deriving therefrom by one or more nucleotide modifications, such as nucleotide substitution, addition or deletion, that results in the same or substantially the same level of expression (e.g. ±20%, such as ±10%, ±5% or ±1%) of the ASAH1 transgene operatively linked thereto.

Of course, other sequences such as a Kozak sequence (e.g. GCCACC) are known to those skilled in the art and are introduced to allow expression of a transgene.

In another particular embodiment, the expression cassette may comprise a further regulatory element located between the ASAH1 ORF and the polyadenylation signal. Representative regulatory elements that may be useful in the present invention include, without limitation, the 3′-untranslated region (3′-UTR) of a gene, such as the 3′-UTR of the ASAH1 gene, the 3′-UTR of the HBB gene, the 3′-UTR of SV40 or the 3′-UTR of the bovine growth hormone. Alternatively, the further regulatory element located between the ASAH1 ORF and the polyadenylation signal is a WPRE sequence, such as the WPRE sequence having the nucleic acid sequence shown in SEQ ID NO:10.

In yet another embodiment, the expression cassette comprises, in this order:
a PGK promoter;
a modified intron 2/exon 3 sequence from the human β globin gene;
an ASAH1 ORF; and
a polyadenylation signal.

In a further particular embodiment, the expression cassette comprises, in this order:
the PGK promoter of SEQ ID NO:3, or a functional variant thereof;
the modified intron 2/exon 3 sequence from the human β globin gene of SEQ ID NO:4, or a functional variant thereof;
an ASAH1 ORF; and
a polyadenylation signal which is the HBB polyadenylation signal of SEQ ID NO:2, or a functional variant thereof.

In a further particular embodiment, the expression cassette comprises, in this order:
the PGK promoter of SEQ ID NO:3, or a functional variant thereof which is at least 99% identical to SEQ ID NO:3;
the modified intron 2/exon 3 sequence from the human β globin gene of SEQ ID NO:4, or a functional variant thereof which is at least 99% identical to SEQ ID NO:4;
an ASAH1 ORF; and
a polyadenylation signal which is the HBB polyadenylation signal of SEQ ID NO:2, or a functional variant thereof which is at least 99% identical to SEQ ID NO:2.

In a further particular embodiment, the expression cassette comprises, in this order:
the PGK promoter of SEQ ID NO:3;
the modified intron 2/exon 3 sequence from the human β globin gene of SEQ ID NO:4, or a functional variant thereof which is at least 99% identical to SEQ ID NO:4;
an ASAH1 ORF; and
a polyadenylation signal which is the HBB polyadenylation signal of SEQ ID NO:2, or a functional variant thereof which is at least 99% identical to SEQ ID NO:2.

In a further particular embodiment, the expression cassette comprises, in this order:
the PGK promoter of SEQ ID NO:3, or a functional variant thereof which is at least 99% identical to SEQ ID NO:3;
the modified intron 2/exon 3 sequence from the human β globin gene of SEQ ID NO:4;
an ASAH1 ORF; and
a polyadenylation signal which is the HBB polyadenylation signal of SEQ ID NO:2, or a functional variant thereof which is at least 99% identical to SEQ ID NO:2.

In a further particular embodiment, the expression cassette comprises, in this order:
the PGK promoter of SEQ ID NO:3, or a functional variant thereof which is at least 99% identical to SEQ ID NO:3;
the modified intron 2/exon 3 sequence from the human β globin gene of SEQ ID NO:4, or a functional variant thereof which is at least 99% identical to SEQ ID NO:4;
a polyadenylation signal which is the HBB polyadenylation signal of SEQ ID NO:2.

In a further particular embodiment, the expression cassette comprises, in this order:
the PGK promoter of SEQ ID NO:3;
the modified intron 2/exon 3 sequence from the human β globin gene of SEQ ID NO:4;
an ASAH1 ORF; and
a polyadenylation signal which is the HBB polyadenylation signal of SEQ ID NO:2, or a functional variant thereof which is at least 99% identical to SEQ ID NO:2.

In a further particular embodiment, the expression cassette comprises, in this order:
the PGK promoter of SEQ ID NO:3;
the modified intron 2/exon 3 sequence from the human β globin gene of SEQ ID NO:4, or a functional variant thereof which is at least 99% identical to SEQ ID NO:4;
an ASAH1 ORF; and
a polyadenylation signal which is the HBB polyadenylation signal of SEQ ID NO:2.

In a further particular embodiment, the expression cassette comprises, in this order:
the PGK promoter of SEQ ID NO:3, or a functional variant thereof which is at least 99% identical to SEQ ID NO:3;
the modified intron 2/exon 3 sequence from the human β globin gene of SEQ ID NO:4;
an ASAH1 ORF; and
a polyadenylation signal which is the HBB polyadenylation signal of SEQ ID NO:2.

In a further particular embodiment, the expression cassette comprises, in this order:
the PGK promoter of SEQ ID NO:3;
the modified intron 2/exon 3 sequence from the human β globin gene of SEQ ID NO:4;
an ASAH1 ORF; and
a polyadenylation signal which is the HBB polyadenylation signal of SEQ ID NO:2.

In a preferred embodiment, the expression cassette comprises a CAG promoter.

In a particular embodiment, of the invention, the expression cassette comprises: in this order:
a CAG promoter;
an ASAH1 ORF; and
a polyadenylation signal.

In a particular embodiment of the invention, the expression cassette comprises: in this order:
a promoter which is the CAG promoter of SEQ ID NO:9, or a functional variant thereof;
an ASAH1 ORF; and
a polyadenylation signal which is the HBB polyadenylation signal of SEQ ID NO:2, or a functional variant thereof.

In a further particular embodiment, the expression cassette comprises, in this order:
a promoter which is the CAG promoter of SEQ ID NO:9, or a functional variant thereof which is at least 99% identical to SEQ ID NO:9;
an ASAH1 ORF; and
a polyadenylation signal which is the HBB polyadenylation signal of SEQ ID NO:2, or a functional variant thereof which is at least 99% identical to SEQ ID NO:2.

In a further particular embodiment, the expression cassette comprises, in this order:
a promoter which is the CAG promoter shown in SEQ ID NO:9;
an ASAH1 ORF; and
a polyadenylation signal which is the HBB polyadenylation signal of SEQ ID NO:2, or a functional variant thereof which is at least 99% identical to SEQ ID NO:2.

In a further particular embodiment, the expression cassette comprises, in this order:
a promoter which is the CAG promoter shown in SEQ ID NO:9, or a functional variant thereof;
an ASAH1 ORF; and
a polyadenylation signal which is the HBB polyadenylation signal of SEQ ID NO:2.

In a further particular embodiment, the expression cassette comprises, in this order:
a promoter which is the CAG promoter shown in SEQ ID NO:9;
an ASAH1 ORF; and
a polyadenylation signal which is the HBB polyadenylation signal of SEQ ID NO:2.

Preferred embodiments of the above described specific expression cassettes include the use of an ASAH1 ORF, wherein the nucleic acid sequence of said ASAH1 ORF comprises or consists of:
the wild-type sequence shown in SEQ ID NO:5, or a functional variant thereof, such as a functional variant which is at least 99% identical to SEQ ID NO:5;
the wild-type sequence shown in SEQ ID NO:6, or a functional variant thereof, such as a functional variant which is at least 99% identical to SEQ ID NO:6;
an optimized sequence consisting of SEQ ID NO:7 or a functional variant thereof, such as a functional variant which is at least 99% identical to SEQ ID NO:7; or
an optimized sequence consisting of SEQ ID NO:8 or a functional variant thereof, such as a functional variant which is at least 99% identical to SEQ ID N0:8.

In a preferred embodiment the nucleic acid sequence of said ASAH1 ORF comprises or consists of:
the wild-type sequence shown in SEQ ID NO:5, or a functional variant thereof, such as a functional variant which is at least 99% identical to SEQ ID NO:5; or
an optimized sequence consisting of SEQ ID NO:7 or SEQ ID NO:8 or a functional variant thereof, such as a functional variant which is at least 99% identical to SEQ ID NO:7

In yet another preferred embodiment the nucleic acid sequence of said ASAH1 ORF comprises or consists of:
the wild-type sequence shown in SEQ ID NO:5; or
an optimized sequence consisting of SEQ ID NO:7 or SEQ ID NO:8.

In yet a further preferred embodiment, the nucleic acid sequence of said ASAH1 ORF consists of:
the wild-type sequence shown in SEQ ID NO:5;
an optimized sequence consisting of SEQ ID NO:7;
an optimized sequence consisting of SEQ ID NO:8.

In a particular embodiment, the nucleic acid sequence of said ASAH1 ORF consists of the wild-type sequence shown in SEQ ID NO:5.

### Recombinant Vectors

To be used as a genome for a rAAV vector, the expression cassette disclosed herein is flanked by AAV 5′-ITR and an AAV 3′-ITR sequences, which are suitable for the packaging of the expression cassette into a rAAV vector.

The human parvovirus Adeno-Associated Virus (AAV) is a dependovirus that is naturally defective for replication, which is able to integrate into the genome of the infected cell to establish a latent infection. AAV vectors have arisen considerable interest as potential vectors for human gene therapy. Among the favorable properties of the virus are its lack of association with any human disease, its ability to infect both dividing and non-dividing cells, and the wide range of cell lines derived from different tissues that can be infected.

In the context of the present invention, the terms “adeno-associated virus” (AAV) and “recombinant adeno-associated virus” (rAAV) are used interchangeably and refer to an AAV whose genome was modified, as compared to a wild-type (wt) AAV genome, by replacement of a part of the wt genome with a transgene of interest. The term “transgene” refers to a gene whose nucleic acid sequence is non-naturally occurring in an AAV genome. In particular, the rAAV vector is to be used in gene therapy. As used herein, the term “gene therapy” refers to the transfer of genetic material (e.g., DNA or RNA) of interest into a host to treat or prevent a genetic or acquired disease or condition.

Recombinant AAVs may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus. Desirable AAV elements for assembly into vectors include the cap proteins, including the vp1, vp2, vp3 and hypervariable regions, the rep proteins, including rep 78, rep 68, rep 52, and rep 40, and the sequences encoding these proteins. These elements may be readily used in a variety of vector systems and host cells.

In the present invention, the capsid of the AAV vector may be derived from a naturally or non-naturally-occurring serotype. In a particular embodiment, the serotype of the capsid of the AAV vector is selected from AAV natural serotypes. Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. A capsid from an artificial AAV serotype may be, without limitation, a chimeric AAV capsid, a recombinant AAV capsid, or a “humanized” AAV capsid.

According to a particular embodiment, the capsid of the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9 and AAV-9 variants (such as AAVhu68), -2G9, -10 such as -cy10 and -rh10, -11, -12, -rh39, -rh43, -rh74, -dj, Anc80L65, LK03, AAV.PHP.B, AAV2i8, clade F AAVHSC, porcine AAV such as AAVpo4 and AAVpo6, and tyrosine, lysine and serine capsid mutants of AAV serotypes. In addition, the capsid of other non-natural engineered variants (such as AAV-spark100), chimeric AAV or AAV serotypes obtained by shuffling, rationale design, error prone PCR, and machine learning technologies can also be useful.

The AAV vector may also comprise a capsid which is a capsid modified with the P1 peptide, such the AAV9P1 capsid (described in Kunze et al., 2018, Glia, 66(2): p. 413; and Weinmann et al., 2020, Nature Communications, 11(1): p. 5432) or the AAVpo1A1 capsid (described in WO2019/207132).

In a particular embodiment, the AAV vector has a naturally occurring capsid, such as an AAV1, AAV2, AAV3, AAV4, AAV5, AAV6, AAV7, AAV8, AAV9, AAV-cy.10, AAVrh.10, AAVrh.39, AAV11, AAV12 and clade F AAVHSC capsids. In a particular embodiment, the rAAV vector is able to cross the blood-brain barrier. Illustrative AAV vector having the ability to cross the blood-brain barrier include, without limitation, vectors comprising an AAV9 capsid, an AAV10 capsid, such as an AAVrh.10 or AAVcy.10 or a clade F AAVHSC (such as AAVHSC7, AAVHSC15 and AAVHSC17) capsid. In yet another embodiment, the AAV vector comprises an AAV9 capsid.

In particular embodiments of the invention, a rAAV vector may comprise an AAV9 or AAV10 capsid (such as an AAVrh.10 capsid or AAVcy.10 capsid, in particular an AAVrh.10 capsid), or any other capsid from an AAV serotype able to cross the blood-brain barrier such as an AAV9P1 capsid, an AAVpo1A1 capsid, an AAVrh.39 capsid or a clade F AAVHSC capsid. In a further embodiment, the AAV vector comprises an AAV9 or AAV10 capsid (such as an AAVrh.10 capsid or AAVcy.10 capsid, in particular an AAVrh.10 capsid), or any other capsid from an AAV serotype able to cross the blood-brain barrier such as an AAVrh.39 capsid or a clade F AAVHSC capsid. In another embodiment, the AAV vector comprises an AAV9 or AAV10 capsid (such as an AAVrh.10 capsid or AAVcy.10 capsid, in particular an AAVrh.10 capsid). In yet another embodiment, the AAV vector comprises an AAV9 capsid.

The genome present within the rAAV vector of the present invention may be single-stranded or self-complementary. In the context of the present invention a “single stranded genome” is a genome that is not self-complementary, i.e. the coding region contained therein has not been designed as disclosed in McCarty et al., 2001 and 2003 (Op. cit) to form an intra-molecular double-stranded DNA template. On the contrary, a “self-complementary AAV genome” has been designed as disclosed in McCarty et al., 2001 and 2003 (Op. cit) to form an intra-molecular double-stranded DNA template.

In a particular embodiment, the rAAV genome is a single stranded genome.

The genome present within the rAAV vector may preferably lack AAV rep and cap genes, and comprises a transgene of interest. Therefore, the AAV genome may comprise a transgene of interest flanked by AAV ITRs. As described above, the transgene of interest is an ASAH1 ORF, comprised in an expression cassette. The ITRs may be derived from any AAV genome, such as an AAV1, AAV2, AAV3, AAV4, AAV5, AAV6, AAV7, AAV8, AAV9, AAVcy.10, AAVrh.10, AAVrh.39, AAV11, AAV12 or clade F AAVHSC genome. In a particular embodiment, the genome of the AAV vector comprises 5′- and 3′-AAV2 ITRs.

Any combination of AAV serotype capsid and ITR may be implemented in the context of the present invention, meaning that the AAV vector may comprise a capsid and ITRs derived from the same AAV serotype, or a capsid derived from a first serotype and ITRs derived from a different serotype than the first serotype. Such a vector with capsid ITRs deriving from different serotypes is also termed a “pseudotyped vector”. More particularly, the pseudotyped rAAV vector can include:
a genome comprising AAV1 5′- and 3′-ITRs, and a capsid selected in the group consisting of an AAV2, AAV3, AAV4, AAV5, AAV6, AAV7, AAV8, AAV9, AAV9P1, AAVpo1A1, AAVrh.10, AAV11, AAV12, AAVrh.39, AAV-PHP.B and AAVHSC capsid;
a genome comprising AAV2 5′- and 3′-ITRs, and a capsid selected in the group consisting of an AAV1, AAV3, AAV4, AAV5, AAV6, AAV7, AAV8, AAV9, AAV9P1, AAVpo1A1, AAVrh.10, AAV11, AAV12, AAVrh.39, AAV-PHP.B and AAVHSC capsid;
a genome comprising AAV3 5′- and 3′-ITRs, and a capsid selected in the group consisting of an AAV1, AAV2, AAV4, AAV5, AAV6, AAV7, AAV8, AAV9, AAV9P1, AAVpo1A1, AAVrh.10, AAV11, AAV12, AAVrh.39, AAV-PHP.B and AAVHSC capsid;
a genome comprising AAV4 5′- and 3′-ITRs, and a capsid selected in the group consisting of an AAV1, AAV2, AAV3, AAV5, AAV6, AAV7, AAV8, AAV9, AAV9P1, AAVpo1A1, AAVrh.10, AAV11, AAV12, AAVrh.39, AAV-PHP.B and AAVHSC capsid;
a genome comprising AAV5 5′- and 3′-ITRs, and a capsid selected in the group consisting of an AAV1, AAV2, AAV3, AAV4, AAV6, AAV7, AAV8, AAV9, AAV9P1, AAVpo1A1, AAVrh.10, AAV11, AAV12, AAVrh.39, AAV-PHP.B and AAVHSC capsid;
a genome comprising AAV6 5′- and 3′-ITRs, and a capsid selected in the group consisting of an AAV1, AAV2, AAV3, AAV4, AAV5, AAV7, AAV8, AAV9, AAV9P1, AAVpo1A1, AAVrh.10, AAV11, AAV12, AAVrh.39, AAV-PHP.B and AAVHSC capsid;
a genome comprising AAV7 5′- and 3′-ITRs, and a capsid selected in the group consisting of an AAV1, AAV2, AAV3, AAV4, AAV5, AAV6, AAV8, AAV9, AAV9P1, AAVpo1A1, AAVrh.10, AAV11, AAV12, AAVrh.39, AAV-PHP.B and AAVHSC capsid;
a genome comprising AAV8 5′- and 3′-ITRs, and a capsid selected in the group consisting of an AAV1, AAV2, AAV3, AAV4, AAV5, AAV6, AAV7, AAV9, AAV9P1, AAVpo1A1, AAVrh.10, AAV11, AAV12, AAVrh.39, AAV-PHP.B and AAVHSC capsid;
a genome comprising AAV9 5′- and 3′-ITRs, and a capsid selected in the group consisting of an AAV1, AAV2, AAV3, AAV4, AAV5, AAV6, AAV7, AAV8, AAV9P1, AAVpo1A1, AAVrh.10, AAV11, AAV12, AAVrh.39, AAV-PHP.B and AAVHSC capsid;
a genome comprising AAVrh.10 5′- and 3′-ITRs, and a capsid selected in the group consisting of an AAV1, AAV2, AAV3, AAV4, AAV5, AAV6, AAV7, AAV8, AAV9, AAV9P1, AAVpo1A1, AAV11, AAV12, AAVrh.39, AAV-PHP.B and AAVHSC capsid;
a genome comprising AAV11 5′- and 3′-ITRs, and a capsid selected in the group consisting of an AAV1, AAV2, AAV3, AAV4, AAV5, AAV6, AAV7, AAV8, AAV9, AAV9P1, AAVpo1A1, AAVrh.10, AAV12, AAVrh.39, AAV-PHP.B and AAVHSC capsid;
a genome comprising AAV12 5′- and 3′-ITRs, and a capsid selected in the group consisting of an AAV1, AAV2, AAV3, AAV4, AAV5, AAV6, AAV7, AAV8, AAV9, AAV9P1, AAVpo1A1, AAVrh.10, AAV11, AAVrh.39 AAV-PHP.B and AAVHSC capsid;
a genome comprising AAVrh.39 5′- and 3′-ITRs, and a capsid selected in the group consisting of an AAV1, AAV2, AAV3, AAV4, AAV5, AAV6, AAV7, AAV8, AAV9, AAV9P1, AAVpo1A1, AAVrh.10, AAV11, AAV12, AAV-PHP.B and AAVHSC capsid;
a genome comprising AAV-PHP.B 5′- and 3′-ITRs, and a capsid selected in the group consisting of an AAV1, AAV2, AAV3, AAV4, AAV5, AAV6, AAV7, AAV8, AAV9, AAV9P1, AAVpo1A1, AAVrh.10, AAV11, AAV12, AAVrh.39 and AAVHSC capsid; or

a genome comprising AAVHSC 5′- and 3′-ITRs, and a capsid selected in the group consisting of an AAV1, AAV2, AAV3, AAV4, AAV5, AAV6, AAV7, AAV8, AAV9, AAV9P1, AAVpo1A1, AAVrh.10, AAV11, AAV12, AAVrh.39 and AAV-PHP.B capsid.

In another embodiment, the pseudotyped rAAV vector can include:
a genome comprising AAV1 5′- and 3′-ITRs, and a capsid selected in the group consisting of an AAV2, AAV3, AAV4, AAV5, AAV6, AAV7, AAV8, AAV9, AAVrh.10, AAV11, AAV12, AAVrh.39, AAV-PHP.B and AAVHSC capsid;
a genome comprising AAV2 5′- and 3′-ITRs, and a capsid selected in the group consisting of an AAV1, AAV3, AAV4, AAV5, AAV6, AAV7, AAV8, AAV9, AAVrh.10, AAV11, AAV12, AAVrh.39, AAV-PHP.B and AAVHSC capsid;
a genome comprising AAV3 5′- and 3′-ITRs, and a capsid selected in the group consisting of an AAV1, AAV2, AAV4, AAV5, AAV6, AAV7, AAV8, AAV9, AAVrh.10, AAV11, AAV12, AAVrh.39, AAV-PHP.B and AAVHSC capsid;
a genome comprising AAV4 5′- and 3′-ITRs, and a capsid selected in the group consisting of an AAV1, AAV2, AAV3, AAV5, AAV6, AAV7, AAV8, AAV9, AAVrh.10, AAV11, AAV12, AAVrh.39, AAV-PHP.B and AAVHSC capsid;
a genome comprising AAV5 5′- and 3′-ITRs, and a capsid selected in the group consisting of an AAV1, AAV2, AAV3, AAV4, AAV6, AAV7, AAV8, AAV9, AAVrh.10, AAV11, AAV12, AAVrh.39, AAV-PHP.B and AAVHSC capsid;
a genome comprising AAV6 5′- and 3′-ITRs, and a capsid selected in the group consisting of an AAV1, AAV2, AAV3, AAV4, AAV5, AAV7, AAV8, AAV9, AAVrh.10, AAV11, AAV12, AAVrh.39, AAV-PHP.B and AAVHSC capsid;
a genome comprising AAV7 5′- and 3′-ITRs, and a capsid selected in the group consisting of an AAV1, AAV2, AAV3, AAV4, AAV5, AAV6, AAV8, AAV9, AAVrh.10, AAV11, AAV12, AAVrh.39, AAV-PHP.B and AAVHSC capsid;
a genome comprising AAV8 5′- and 3′-ITRs, and a capsid selected in the group consisting of an AAV1, AAV2, AAV3, AAV4, AAV5, AAV6, AAV7, AAV9, AAVrh.10, AAV11, AAV12, AAVrh.39, AAV-PHP.B and AAVHSC capsid;
a genome comprising AAV9 5′- and 3′-ITRs, and a capsid selected in the group consisting of an AAV1, AAV2, AAV3, AAV4, AAV5, AAV6, AAV7, AAV8, AAVrh.10, AAV11, AAV12, AAVrh.39, AAV-PHP.B and AAVHSC capsid;
a genome comprising AAVrh.10 5′- and 3′-ITRs, and a capsid selected in the group consisting of an AAV1, AAV2, AAV3, AAV4, AAV5, AAV6, AAV7, AAV8, AAV9, AAV11, AAV12, AAVrh.39, AAV-PHP.B and AAVHSC capsid;
a genome comprising AAV11 5′- and 3′-ITRs, and a capsid selected in the group consisting of an AAV1, AAV2, AAV3, AAV4, AAV5, AAV6, AAV7, AAV8, AAV9, AAVrh.10, AAV12, AAVrh.39, AAV-PHP.B and AAVHSC capsid;
a genome comprising AAV12 5′- and 3′-ITRs, and a capsid selected in the group consisting of an AAV1, AAV2, AAV3, AAV4, AAV5, AAV6, AAV7, AAV8, AAV9, AAVrh.10, AAV11, AAVrh.39 AAV-PHP.B and AAVHSC capsid;
a genome comprising AAVrh.39 5′- and 3′-ITRs, and a capsid selected in the group consisting of an AAV1, AAV2, AAV3, AAV4, AAV5, AAV6, AAV7, AAV8, AAV9, AAVrh.10, AAV11, AAV12, AAV-PHP.B and AAVHSC capsid;
a genome comprising AAV-PHP.B 5′- and 3′-ITRs, and a capsid selected in the group consisting of an AAV1, AAV2, AAV3, AAV4, AAV5, AAV6, AAV7, AAV8, AAV9, AAVrh.10, AAV11, AAV12, AAVrh.39 and AAVHSC capsid; or
a genome comprising AAVHSC 5′- and 3′-ITRs, and a capsid selected in the group consisting of an AAV1, AAV2, AAV3, AAV4, AAV5, AAV6, AAV7, AAV8, AAV9, AAVrh.10, AAV11, AAV12, AAVrh.39 and AAV-PHP.B capsid.

In another embodiment, the pseudotyped rAAV vector can include:
a genome comprising AAV1 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV2 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV3 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV4 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV5 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV6 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV7 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV8 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV9 5′- and 3′-ITRs, and a capsid selected in the group consisting of an AAV9P1, AAVpo1A1, AAVrh.10, AAVrh.39, AAV-PHP.B and AAVHSC capsid;
a genome comprising AAVrh.10 5′- and 3′-ITRs, and a capsid selected in the group consisting of an AAV9, AAV9P1, AAVpo1A1, AAVrh.10, AAVrh.39, AAV-PHP.B and AAVHSC capsid; or
a genome comprising AAV11 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAV12 5′- and 3′-ITRs, and a capsid selected in the group consisting of an AAV9, AAV9P1, AAVpo1A1, AAVrh.10, AAVrh.39, AAV-PHP.B and AAVHSC capsid;
a genome comprising AAVrh.39 5′- and 3′-ITRs, and a capsid selected in the group consisting of an AAV9, AAV9P1, AAVpo1A1, AAVrh.10, AAV-PHP.B and AAVHSC capsid;
a genome comprising AAV-PHP.B 5′- and 3′-ITRs, and a capsid selected in the group consisting of an AAV9, AAV9P1, AAVpo1A1, AAVrh.10, AAVrh.39 and AAVHSC capsid;
a genome comprising AAVHSC 5′- and 3′-ITRs, and a capsid selected in the group consisting of an AAV9, AAV9P1, AAVpo1A1, AAVrh.10, AAVrh.39 and AAV-PHP.B.

In another embodiment, the pseudotyped rAAV vector can include:
a genome comprising AAV1 5′- and 3′-ITRs, and a capsid selected in the group consisting of an AAV9, AAVrh.10, AAVrh.39, AAV-PHP.B and AAVHSC capsid;
a genome comprising AAV2 5′- and 3′-ITRs, and a capsid selected in the group consisting of an AAV9, AAVrh.10, AAVrh.39, AAV-PHP.B and AAVHSC capsid;
a genome comprising AAV3 5′- and 3′-ITRs, and a capsid selected in the group consisting of an AAV9, AAVrh.10, AAVrh.39, AAV-PHP.B and AAVHSC capsid;
a genome comprising AAV4 5′- and 3′-ITRs, and a capsid selected in the group consisting of an AAV9, AAVrh.10, AAVrh.39, AAV-PHP.B and AAVHSC capsid;
a genome comprising AAVS 5′- and 3′-ITRs, and a capsid selected in the group consisting of an AAV9, AAVrh.10, AAVrh.39, AAV-PHP.B and AAVHSC capsid;
a genome comprising AAV6 5′- and 3′-ITRs, and a capsid selected in the group consisting of an AAV9, AAVrh.10, AAVrh.39, AAV-PHP.B and AAVHSC capsid;
a genome comprising AAV7 5′- and 3′-ITRs, and a capsid selected in the group consisting of an AAV9, AAVrh.10, AAVrh.39, AAV-PHP.B and AAVHSC capsid;
a genome comprising AAV8 5′- and 3′-ITRs, and a capsid selected in the group consisting of an AAV9, AAVrh.10, AAVrh.39, AAV-PHP.B and AAVHSC capsid;
a genome comprising AAV9 5′- and 3′-ITRs, and a capsid selected in the group consisting of an AAVrh.10, AAVrh.39, AAV-PHP.B and AAVHSC capsid;
a genome comprising AAVrh.10 5′- and 3′-ITRs, and a capsid selected in the group consisting of an AAV9, AAVrh.10, AAVrh.39, AAV-PHP.B and AAVHSC capsid; or
a genome comprising AAV11 5′- and 3′-ITRs, and a capsid selected in the group consisting of an AAV9, AAVrh.10, AAVrh.39, AAV-PHP.B and AAVHSC capsid;
a genome comprising AAV12 5′- and 3′-ITRs, and a capsid selected in the group consisting of an AAV9, AAVrh.10, AAVrh.39, AAV-PHP.B and AAVHSC capsid;
a genome comprising AAVrh.39 5′- and 3′-ITRs, and a capsid selected in the group consisting of an AAV9, AAVrh.10, AAV-PHP.B and AAVHSC capsid;
a genome comprising AAV-PHP.B 5′- and 3′-ITRs, and a capsid selected in the group consisting of an AAV9, AAVrh.10, AAVrh.39 and AAVHSC capsid;
a genome comprising AAVHSC 5′- and 3′-ITRs, and a capsid selected in the group consisting of an AAV9, AAVrh.10, AAVrh.39 and AAV-PHP.B.

In a particular embodiment, the pseudotyped rAAV vector includes a genome, in particular a single-stranded genome, comprising AAV2 5′- and 3′-ITRs, and a capsid selected in the group consisting of an AAV1, AAV3, AAV4, AAV5, AAV6, AAV7, AAV8, AAV9, AAV9P1, AAVpo1A1, AAVrh.10, AAV11, AAV12, AAVrh.39, AAV-PHP.B and AAVHSC capsid. In a further embodiment, the pseudotyped rAAV vector includes a genome, in particular a single-stranded genome, comprising AAV2 5′- and 3′-ITRs, and a capsid selected in the group consisting of an AAV1, AAV3, AAV4, AAV5, AAV6, AAV7, AAV8, AAV9, AAVrh.10, AAV11, AAV12, AAVrh.39, AAV-PHP.B and AAVHSC capsid. In another particular embodiment, the pseudotyped rAAV vector includes a genome, in particular a single-stranded genome, comprising AAV2 5′- and 3′-ITRs, and a capsid selected in the group consisting of an AAV9, AAVrh.10, AAVrh.39, AAV-PHP.B and AAVHSC capsid. In yet another particular embodiment, the pseudotyped rAAV vector includes a genome, in particular a single-stranded genome, comprising AAV2 5′- and 3′-ITRs, and an AAV9 capsid.

In another aspect, the invention provides DNA plasmids comprising rAAV genomes of the invention. Production of rAAV requires that the following components are present within a single cell (denoted herein as a packaging cell): a rAAV genome, AAV rep and cap genes separate from (i.e., not in) the rAAV genome, and helper virus functions. Production of pseudotyped rAAV is disclosed in, for example, WO 01/83692. Production may implement transfection a cell with two, three or more plasmids. For example three plasmids may be used, including: (i) a plasmid carrying a Rep/Cap cassette, (ii) a plasmid carrying the rAAV genome (i.e. a transgene flanked with AAV ITRs) and (iii) a plasmid carrying helper virus functions (such as adenovirus helper functions). In another embodiment, a two-plasmid system may be used, comprising (i) a plasmid comprising Rep and Cap genes, and helper virus functions, and (ii) a plasmid comprising the rAAV genome.

In a further aspect, the invention relates to a plasmid comprising the expression cassette described above. This plasmid may be introduced in a cell for producing a rAAV vector according to the invention by providing the rAAV genome to said cell.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are incorporated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62: 1963 (1988); and Lebkowski et al., 1988 Mol. Cell. Biol., 7:349 (1988); Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13: 1244-1250; Paul et al. (1993) Human Gene Therapy 4:609-615; Clark et al. (1996) Gene Therapy 3: 1124-1132; U.S. Pat. Nos. 5,786,211; 5,871,982; and 6,258,595. The invention thus also provides packaging cells that produce infectious rAAV. In one embodiment packaging cells may be stably transformed cancer cells such as HeLa cells, HEK293 cells, HEK 293T, HEK293vc and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et ah, Hum. Gene Ther., 10(6): 1031-1039 (1999); Schenpp and Clark, Methods Mol. Med., 69: 427-443 (2002); U.S. Pat. No. 6,566,118 and WO 98/09657.

In another aspect, the invention provides compositions comprising a rAAV disclosed in the present application. Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

In another aspect, the invention relates to an expression cassette comprising a ASAH1 ORF and the regulatory elements (such as promoters, introns, 3′-UTRs and polyadenylation signals) as described above, as they are disclosed in each of their broad and specific embodiments.

As shown above, the expression cassette can be introduced into an AAV vector. In alternative embodiments, the expression cassette of the invention can be introduced into another vector, such as another recombinant viral vector or non-viral vector. Therefore, the invention also relates to a vector comprising the expression cassette of the invention, said vector being suitable for gene therapy to express the ASAH1 ORF into target cells of interest for the treatment of an acid ceramidase deficiency of other diseases presenting decreased levels of acid ceramidase activity. In a particular embodiment, the vector is a non-viral vector such as a plasmid, a nanoparticle, a lipid nanoparticle or a liposome. In another embodiment, the vector is a recombinant viral vector such as a rAAV vector as described above, or another viral vector, such as an adenovirus. In a particular embodiment, the recombinant viral vector comprising the expression cassette of the invention is not a lentivirus.

### Therapeutic Uses of the Invention

The invention also relates to a vector, such as a rAAV vector, as disclosed herein, for use in therapy.

Thanks to the present invention, the ASAH1 ORF may be delivered to a subject in need thereof. A subject in need thereof may be a subject having an acid ceramidase deficiency or a disease related or presenting decreased levels of acid ceramidase activity or increased levels of ceramide as compared to normal levels. In a particular embodiment, the disease presenting increased levels of ceramide is cystic fibrosis, some retinopathies (such as retinitis pigmentosa (RP26)) and inherited blindness.

In a preferred embodiment, the rAAV vector of the invention is for use in a method for the treatment of an acid ceramidase deficiency, such as cystic fibrosis, FD or SMA-PME. More particularly, the rAAV vector of the invention is for use in a method for the treatment of FD or SMA-PME. In a particular embodiment, the rAAV vector of the invention is for use in a method for the treatment of SMA-PME. In a particularly preferred embodiment, the vector of the invention, in particular the rAAV vector of the invention, is for use in the treatment of FD.

In a particular embodiment, the vector of the invention, in particular the rAAV vector of the invention, is for use in the treatment of at least one neural manifestation and/or at least one peripheral manifestation of FD.

Neurological manifestations of FD include central nervous system (CNS) manifestations and peripheral nervous system (PNS) manifestations.

CNS manifestations of FD include seizures, developmental delay, intellectual disability, due to hydrocephalus and brain atrophy storage pathology in brain stem, cerebral cortex and cerebellum, hypotonia, muscle weakness and atrophy due to storage pathology in the anterior part of the spinal cord or muscle weakness and atrophy due to storage pathology in the anterior part of the spinal cord. CNS manifestations of FD may also include in other locations of the spinal cord.

PNS manifestations of FD include hypotonia, muscle weakness and atrophy leading to wheelchair due to storage pathology in Schwann cells of the peripheral nervous.

Peripheral manifestations include:
subcutaneous lipogranulomata;
joint pain;
hoarseness of the voice;
hematological manifestations such increased leukocyte count and erythrocyte sedimentation rate, elevated plasma chitotriosidase and C-reactive protein (CRP), enlarged lymph nodes, anemia, thrombocytopenia, presence of nucleated red blood cells;
pulmonary findings: one of the more common signs of the disease in both classic and attenuated forms of FD: sternal retraction, expiratory stridor, aphonia, difficulties in breathing;
ophthalmic findings: most common sign is a cherry red spot, retinal opacification, corneal opacities, macular degeneration;
gastrointestinal manifestations: persistent diarrhea;
hepatic findings: hepatomegaly, cholestatic jaundice, ascites, livers fibrosis and elevated liver enzymes;
spleen: splenomegaly;
cartilage and bone findings: joint erosion, juxta-articular bone erosion and demineralization, osteoporosis, peripheral osteolysis with shortened fingers and toes; and
dermatological manifestations: subcutaneous nodules, skin lesions and plaques, hyperketatosis, keloids.

In a particular embodiment, the rAAV vector of the invention is for use in the treatment of at least one peripheral manifestation of FD. In a further particular embodiment of the invention, the rAAV vector of the invention is for use in the treatment of hematological manifestations of FD. In yet a further embodiment, the vector, in particular the rAAV vector of the invention, of the invention is for use in the treatment of neurological, spleen, lung or hematological manifestations of FD. In yet a further embodiment, the vector of the invention, in particular the rAAV vector of the invention is for use in the treatment of neurological, spleen, lung and hematological manifestations of FD.

In a preferred embodiment, the vector for use according to the invention is a rAAV vector comprising a genome as defined above, such as a single-stranded genome. In yet another embodiment, the vector for use according to the invention may be an AAV9, AAV9P1, AAVpo1A1, AAVrh.10, AAVrh.39, AAV-PHP.B or clade F AAVHSC vector, preferably an AAV9, AAVrh.10, AAVrh.39, AAV-PHP.B or clade F AAVHSC vector, preferably an AAV9 or AAVrh.10 vector, more preferably an AAV9 vector, comprising a genome as defined above, such as a single-stranded genome.

The vector for use according to the invention may be administered locally with or without systemic co-delivery. In the context of the present invention, local administration denotes an administration into the cerebrospinal fluid of the subject, such as via an intrathecal injection of the rAAV vector. In some embodiments, an effective amount of the vector is administrated by intracerebral administration. In some embodiments, the vector may be administrated by intrathecal administration or by intracerebral administration. In some embodiment the vector may be administered by a combined intrathecal and/or intracerebral and/or peripheral (such as a vascular, for example intravenous or intra-arterial, in particular intravenous) administration.

As used herein the term “intrathecal administration” refers to the administration of a vector according to the invention, or a composition comprising a vector of the invention, into the spinal canal. For example, intrathecal administration may comprise injection in the cervical region of the spinal canal, in the thoracic region of the spinal canal, or in the lumbar region of the spinal canal. Typically, intrathecal administration is performed by injecting an agent, e.g., a composition comprising a vector of the invention, into the subarachnoid cavity (subarachnoid space) of the spinal canal, which is the region between the arachnoid membrane and pia mater of the spinal canal. The subarachnoid space is occupied by spongy tissue consisting of trabeculae (delicate connective tissue filaments that extend from the arachnoid mater and blend into the pia mater) and intercommunicating channels in which the cerebrospinal fluid is contained. In some embodiments, intrathecal administration is not administration into the spinal vasculature. In certain embodiment the intrathecal administration is in the lumbar region of the subject

As used herein, the term “intracerebral administration” refers to administration of an agent into and/or around the brain. Intracerebral administration includes, but is not limited to, administration of an agent into the cerebrum, medulla, pons, cerebellum, intracranial cavity, and meninges surrounding the brain. Intracerebral administration may include administration into the dura mater, arachnoid mater, and pia mater of the brain. Intracerebral administration may include, in some embodiments, administration of an agent into the cerebrospinal fluid (CSF) of the subarachnoid space surrounding the brain. Intracerebral administration may include, in some embodiments, administration of an agent into ventricles of the brain/forebrain, e.g., the right lateral ventricle, the left lateral ventricle, the third ventricle, the fourth ventricle. In some embodiments, intracerebral administration is not administration into the brain vasculature.

In some embodiments, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some embodiments, a microinjection pump is used to deliver a composition comprising a vector of the invention. In some embodiments, the infusion rate of the composition is in a range of 1 μl/minute to 100 μl/minute. As will be appreciated by the skilled artisan, infusion rates will depend on a variety of factors, including, for example, species of the subject, age of the subject, weight/size of the subject, the kind of vector (i.e. plasmid or viral vector, type of viral vector, serotype of the vector in case of a rAAV vector), dosage required, intracerebral region targeted, etc. Thus, other infusion rates may be deemed by a skilled artisan to be appropriate in certain circumstances.

Furthermore, thanks to the capacity to cross the blood-brain barrier elicited by certain rAAV vectors (e.g. rAAV9, rAAVrh.10, rAAVrh.39 or rAAVHSC vector) administration via a systemic route may be considered. Accordingly, methods of administration of the rAAV vector include but are not limited to, intramuscular, intraperitoneal, intravascular (e.g. intravenous or intra-arterial), subcutaneous, intranasal, epidural, and oral routes. In a particular embodiment, the systemic administration is an intravascular injection of the rAAV vector, in particular an intravenous injection.

In yet another particular embodiment, the rAAV vector of the invention is a vector having the capacity to cross the blood-brain barrier, such as a rAAV9, AAV9P1, AAVpo1A1, rAAVrh.10, rAAVrh.39 or rAAVHSC vector, in particular such as a rAAV9, rAAVrh.10, rAAVrh.39 or rAAVHSC vector (in particular a rAAV9 or rAAV10 vector, more particularly a rAAV9 vector), the rAAV vector is administered intravascularly (in particular intravenously), and the disease to be treated is FD.

In a further embodiment, the rAAV vector of the invention is a vector having the capacity to cross the blood-brain barrier, such as a rAAV9, AAV9P1, AAVpo1A1, rAAVrh.10, rAAVrh.39 or rAAVHSC vector, in particular such as a rAAV9, rAAVrh.10, rAAVrh.39 or rAAVHSC vector (in particular a rAAV9 or rAAV10 vector, more particularly a rAAV9 vector), the rAAV vector is administered both intravascularly (in particular intravenously) and in the CSF (in particular intracerebroventricularly or intrathecal), and the disease to be treated is FD.

In a particular embodiment, the vector is administered into the cerebrospinal fluid, in particular by intrathecal injection. In a particular embodiment, the patient is put in the Trendelenburg position after intrathecal delivery of an rAAV vector.

The amount of the vector of the invention which will be effective in the treatment of FD or SMA-PME can be determined by standard clinical techniques. In addition, in vivo and/or in vitro assays may optionally be employed to help predict optimal dosage ranges. The dosage of the vector of the invention administered to the subject in need thereof will vary based on several factors including, without limitation, the specific type or stage of the disease treated, the subject's age or the level of expression necessary to obtain the therapeutic effect. One skilled in the art can readily determine, based on its knowledge in this field, the dosage range required based on these factors and others. Typical doses of AAV vectors are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg or at least 1×10¹⁵ vg/kg.

Furthermore, the vector for use according to the invention, in particular a rAAV as disclosed herein, can be used in combination with another therapy. Illustrative, non-limiting embodiments include the following ones, wherein the subject in need thereof may receive:
the recombinant vector according to the invention, such a rAAV of the invention, and a hematopoietic stem cell transplant;
the recombinant vector according to the invention, such a rAAV of the invention, and a hematopoietic stem cell transplant, wherein the hematopoietic stem cells have been transduced with a vector, such as a lentiviral vector, carrying a ASAH1 ORF; or
the recombinant vector according to the invention, such a rAAV of the invention, administered in the CSF (such as by the intracerebroventricular route) and a recombinant ASAH1 protein administered as an ERT via a peripheral route, such as by intravascular administration, more particularly an intravenous administration.

Other illustrative embodiments of combination therapies comprising the recombinant vector according to the invention include, without limitation:
a rAAV vector of the invention and a lentiviral vector encoding an ASAH1 ORF;
a rAAV vector of the invention and a recombinant ASAH1 protein administered as an ERT; and
a rAAV vector of the invention and any other substance, compound or composition suitable for the treatment of an acid ceramidase deficiency.

### EXAMPLES

### Materials and Methods

### Vector Production

A single stranded serotype-9 AAV vector carrying the wild-type ASAH1 ORF (SEQ ID NO:5) under the ubiquitous CAG promoter was produced by using a three-plasmid transfection system in HEK293 cells in suspension culture and purified by affinity chromatography. The vector titer (viral genomes/mL) was determined by qPCR.

A single stranded serotype-9 AAV vector carrying the wild-type ASAH1 ORF (SEQ ID NO:5) or an optimized ASAH1 ORF (SEQ ID NO:7 or SEQ ID NO:8) under the ubiquitous PGK promoter (SEQ ID NO:3), with a modified intron 2/exon 3 sequence from the human β globin gene (SEQ ID NO:4) downstream of the promoter, was produced by using a three-plasmid transfection system in HEK293 cells in suspension culture and purified by affinity chromatography. The vector titer (viral genomes/mL) was determined by qPCR.

### Mouse Genotyping and Vector Injection

Homozygous Asah1^{P361R/P361R} mutant mice were obtained by crossing heterozygous Asah1^{P361R/+} mice (Alayoubi et al. 2013; EMBO Mol Med; 5; p. 827). Litters were genotyped at birth or at fifteen days of life depending on the injection protocol. Mice were kept under a 12-hour light 12-hour dark cycle and fed with a standard diet, food and water ad libitum. Care and manipulation of mice were performed in accordance with national and European legislations on animal experimentation and approved by the institutional ethical committee.

For genotyping, we performed a PCR from genomic DNA and used the following primers: forward 5′(CAGAAGGTATGCGGCATCGTCATAC)3′ (SEQ ID NO:11) and reverse 5′(AGGGCCATACAGAGAAACCCTGTCTC)3′ (SEQ ID NO:12) for wild type Asah1 allele amplification, and forward 5′(TCAAGGCTTGACTTTGGGGCAC)3′ (SEQ ID NO:13) and reverse: 5′(GCTGGACGTAAACTCCTCTTCAGACC)3′ (SEQ ID NO:14) for Asah1 knock-in P361R allele amplification.

Mutant Asah^{P361R/P361R} mice received either 5×10¹³ vg/kg of vector intravenously or 1×10¹³ vg/kg of vector by intracerebroventricular injection at 3 weeks of age or at birth (P0), respectively. Control littermates received equivalent volumes of saline (PBS-MK, 1 mM MgCl2, 2.5 mM KCl). Survival, general appearance, body weight and muscle strength were monitored twice per week. Four or ten weeks after vector injection, mice were anesthetized with a ketamine/xylazene cocktail to collect peripheral blood, followed by cervical dislocation and tissue dissection. The weight of selected organs and muscles was measured before tissue cryopreservation.

### Immunoblot Analysis

Four weeks post-intravenous injection proteins were extracted from tissues using a RIPA lysis buffer according to the manufacturer's protocol (Fisher) supplied with a protease inhibitors cocktail (Complete Mini, Roche Applied Science). Protein samples (100 μg and 50 μg) were separated by SDS-PAGE electrophoresis (1.0 mm, 4-12% gradient Novex NuPAGE Bis-Tris Gel, Life Technologies) and transferred onto a Protran Premium nitrocellulose membrane (GE Healthcare). Membranes were probed with a polyclonal antibody against the C-terminus of human ASAH1 (Sigma) to detect the β subunit, a monoclonal antibody against human ASAH1 (BD Science) to detect the a subunit and a rabbit polyclonal antibody against GAPDH (Millipore) as internal control. After incubation with the corresponding secondary IRDye-680CW or -800CW-conjugated antibodies (1:10,000, LI-COR Biosciences), infrared fluorescence was read on an Odyssey Imaging System (LI-COR Biosciences). Band intensities were measured using Odyssey application software (LI-COR Biosciences, Image Studio Lite, 4.0 Version).

### Functional Evaluation

The four limb-hanging test or Kondziella's inverted screen test represents a method to assess muscle strength in mice. The grid was set at a height of about 35 cm, and a soft bedding was placed underneath to protect the mice from harm in case they fell off the grid. We quantified the holding time of mice aged between 29 to 90 days once a week (3 times/session, 180 sec of maximum holding). The test was performed based on the Treat-NMD guidelines (protocol number DMD_M.2.1.005).

### Blood Analysis

Four weeks after intravenous injection of the vector or saline in mice, blood was collected retro-orbitally into tubes containing an anticoagulant citrate solution. Blood samples were analyzed for standard hematological parameters, including white blood cell (WBC, lymphocytes, monocytes and granulocytes), red blood cell or erythrocytes (RBC) and platelet counts using a MS9.3 counter (Schloessing Melet, France).

### Results

The expression level of acid ceramidase α and β subunits (ASAH1) derived from each expression cassette described in the materials and methods section was evaluated in spinal cord, heart and tibialis anterior (TA) 4 weeks after intravenous injection of 5×10¹³vg/kg of serotype 9 vectors in wild type mice. The level of acid ceramidase was similar in tissues of mice injected with constructs containing a wild-type or optimized ASAH1 ORFs under the control of the PGK promoter, and higher with construct containing the wild-type ASAH1 ORF under the control of the CAG promoter. This shows also that although expression from the PGK promoter was effective, the CAG promoter leads to a stronger ASAH1 expression than the PGK promoter in these tissues. The following results were thus obtained with the construct containing the CAG promoter.

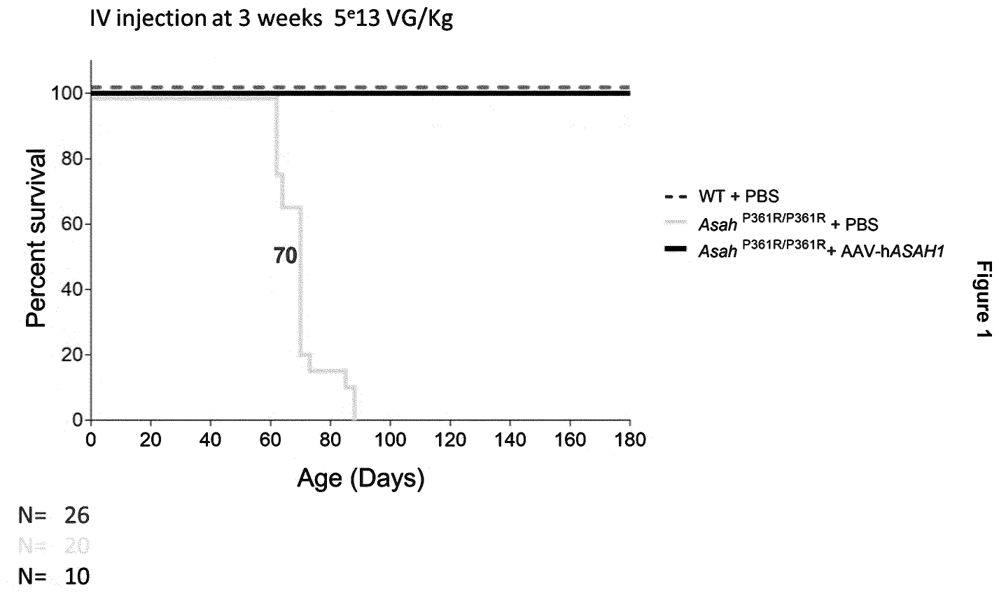

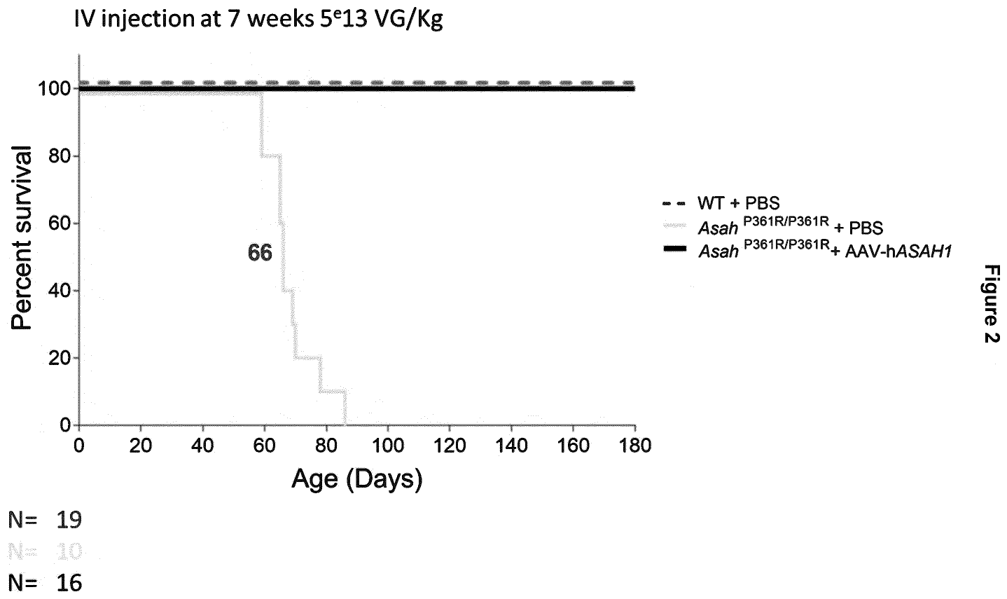

As shown in FIG. 1, intravenous administration at 3 weeks of age of the AAV9-CAG-hASAH1 vector at 5×10¹³ vg/kg prolonged the survival of all treated Asah^{P361R/P361R} mice up to at least 180 days of age compared to untreated mutant mice that died at the mean age of 70 days (n=26 WT+PBS, n=20 Asah^{P361R/P361R}+PBS and n=10 Asah^{P361R/P361R}+AAV-hASAH1 mice at injection). As shown in FIG. 2, a single intravenous injection of the AAV9-CAG-hASAH1 (5×10¹³ vg/kg) at 7 weeks of life prolonged the survival of all treated Asah^{P361R/P361R} mice compared to untreated mutant mice that died at the mean age of 66 days (n=19 WT+PBS, n=10 Asah^{P361R/P361R}+PBS and n=16 Asah^{P361R/P361R}+AAV-hASAH1 mice at injection).

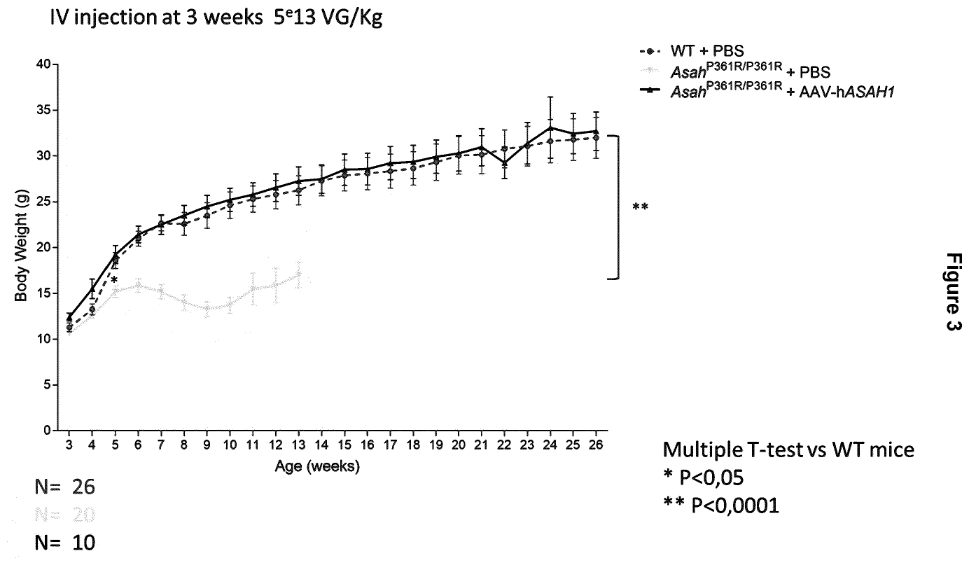

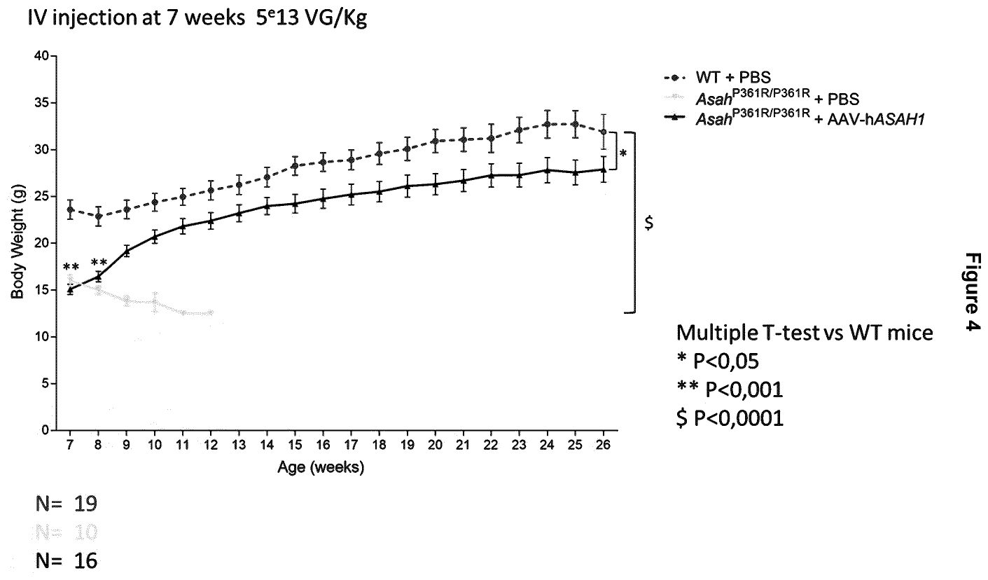

In addition, FIG. 3 shows that body weight of mutant Asah^{P361R/P361R} mice treated intravenously at three weeks of age (early symptomatic stage) with 5×10¹³ vg/kg of AAV9-CAG-hASAH1 increased progressively over time and was comparable to wild type littermates. Whereas untreated mutant mice had a defect in growth starting at around 5 weeks of age (n=26 WT+PBS, n=20 Asah^{P361R/P361R}+PBS and n=10 Asah^{P361R/P361R}+AAV-hASAH1 mice at injection). Furthermore, FIG. 4 shows the increased body weight rescue of mutant Asah^{P361R/P361R} mice treated intravenously at seven weeks of age (post-symptomatic stage) with AAV9-CAG-hASAH1. The weight evolution shows a constant increase over time, whereas untreated mutant mice present a decrease in growth until 12 weeks of age (n=19 WT+PBS, n=10 Asah^{P361R/P361R}+PBS and n=16 Asah^{P361R/P361R}+AAV-hASAH1 mice at injection).

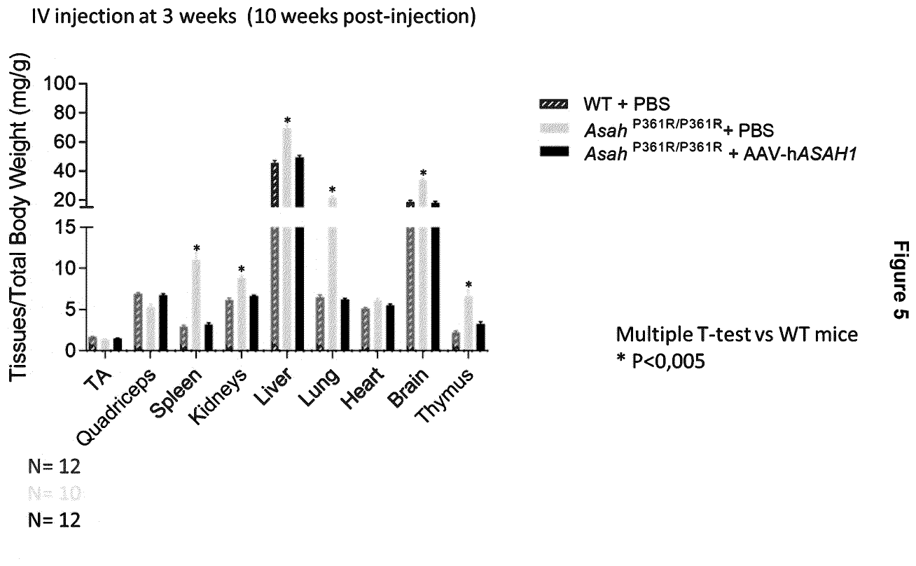

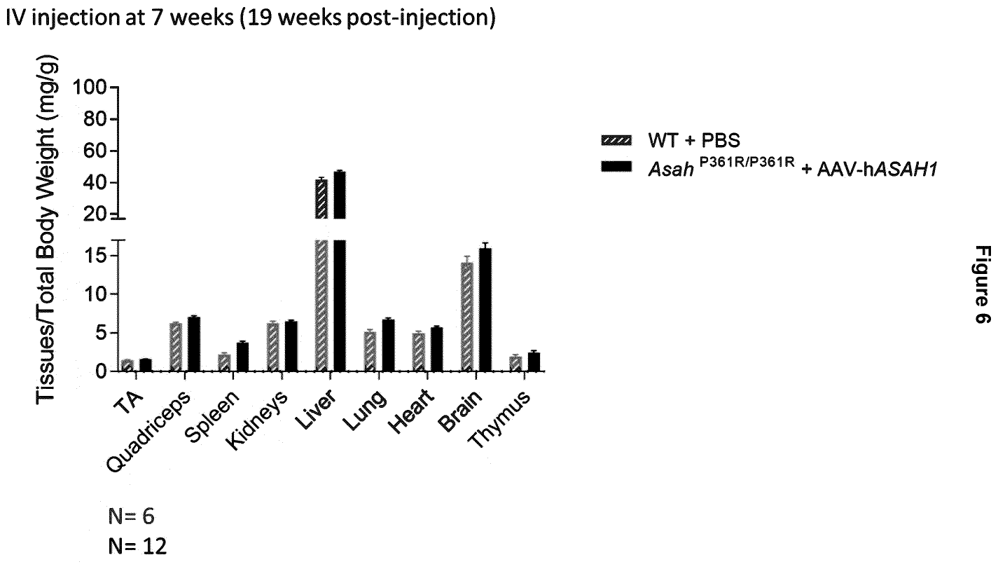

Assessment of disease phenotype in Asah^{P361R/P361R} mice by organs examination reveals (FIG. 5) the enlargement of several peripheral tissues such as spleen, kidney, liver, lung and thymus compared to 13 weeks old control and injected mice. Intravenous injection of ssAAV9-CAG-hASAH1 at 3 weeks of age normalized tissues weight to wild type levels after ten weeks of treatment. Central nervous system is also affected with a higher brain weight compare to control tissues: AAV9-CAG-hASAH1 injection corrects the phenotype to normal size. The ratio of tissue weight over total body weight (mg/g) was also assessed in mice receiving intravenous injection of the vector at 7 weeks of age (FIG. 6). Normalisation of tissues weight to wild type levels was observed, after 19 weeks of treatment.

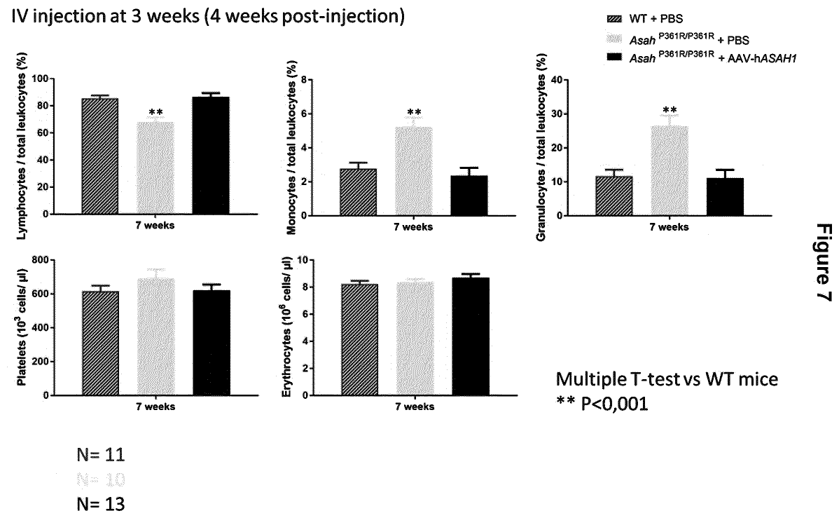

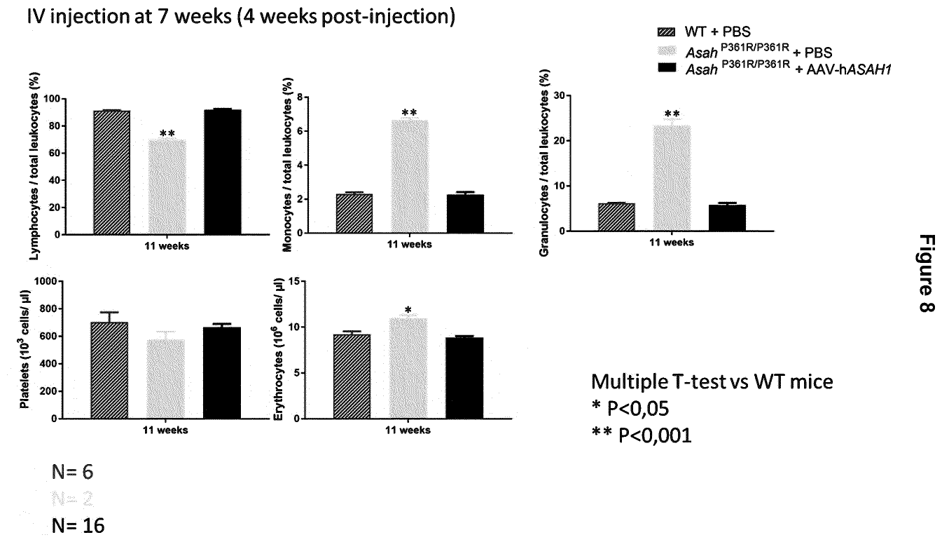

The effect of the administration of the AAV9-CAG-hASAH1 vector on hematopoiesis was also evaluated (FIGS. 7 and 8). As shown in FIG. 7, untreated Asah^{P361R/P361R} mice (N=10) presented altered haematopoiesis compared to wild type mice (N=11) at 7 weeks of age. Intravenous injection of AAV9-CAG-hASAH1 at 3 weeks of age restored the number of lymphocytes, monocytes and granulocytes in peripheral blood after 4 weeks of treatment (N=13). Furthermore, as shown in FIG. 8, systemic administration of AAV9-CAG-hASAH1 at 7 weeks of age corrected altered blood features of Asah^{P361R/P361R} mice (N=16) to wild type levels (N=6). Blood cells count was performed 4 weeks after injection at 11 weeks of life. Not treated Asah^{P361R/P361R} showed greater haematopoiesis alteration (N=2).

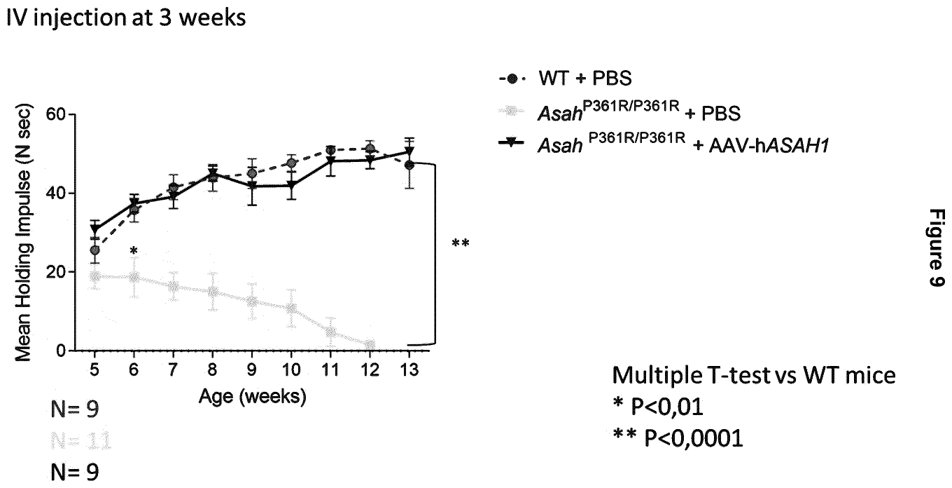

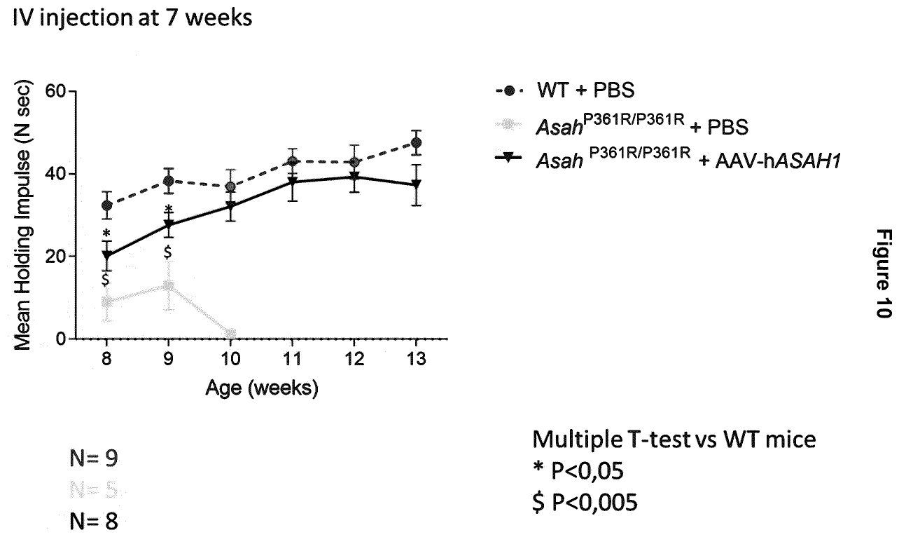

The muscle strength of mice treated intravenously was also evaluated (FIGS. 9 and 10). As shown in FIG. 9, muscle strength of 3 weeks-injected Asah^{P361R/P361R} mice was comparable to wild type animals over the study period as assessed by using the four limb-hanging test, whereas the strength of untreated mutant mice decreased over time (n=9 WT+PBS, n=11 Asah^{P361R/P361R}+PBS and n=9 Asah^{P361R/P361R}+AAV-hASAH1 mice at injection).

FIG. 10 shows that injection of ssAAV9-CAG-hASAH1 at post-symptomatic stage of disease (7 weeks) restored muscle strength of injected Asah^{P361R/P361R} mice to wild type score over the study period (4 to 13 weeks of life), whereas the strength of untreated mutant mice decreased over time (n=9 WT+PBS, n=5 Asah^{P361R/P361R}+PBS and n=8 Asah^{P361R/P361R}+AAV-hASAH1 mice at injection). Results are expressed as the mean±SEM. The differences between groups were analyzed by Multiple T-test (* P<0,05; $ P<0,005).

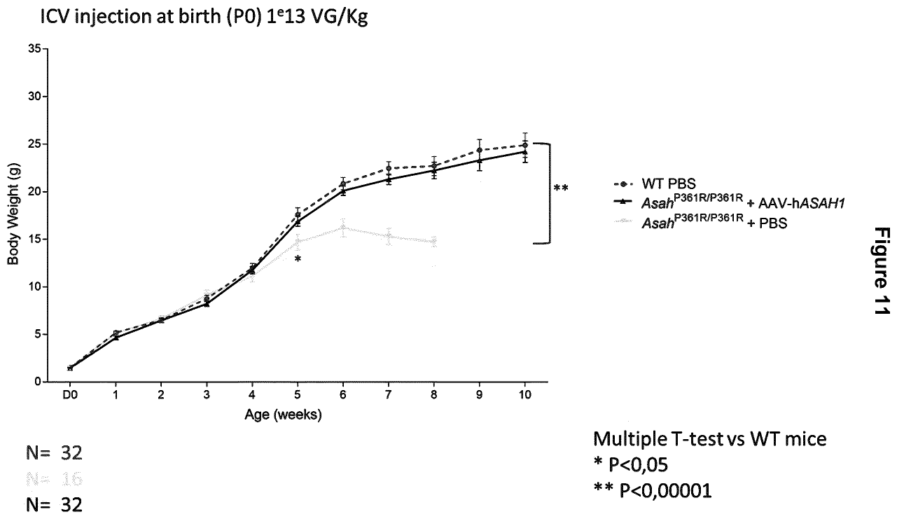

In addition, FIG. 11 shows that the body weight of mutant Asah^{P361R/P361R} mice treated at birth (P0) with 1×10¹³ vg/kg of AAV9-CAG-hASAH1 by intracerebroventricular (ICV) injection increased progressively over time and was comparable to wild type littermates, whereas untreated mutant mice had a growth defect starting at around 5 weeks of age (n=32 WT+PBS, n=16 Asah^{P361R/P361R}+PBS and n=32 Asah^{P361R/P361R}+AAV-hASAH1 mice at injection).

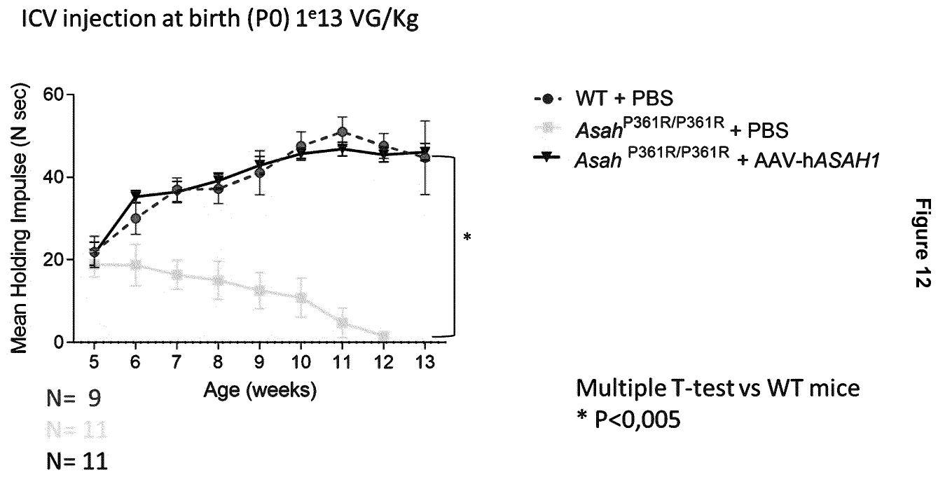

At last, FIG. 12 shows that the muscle strength of Asah^{P361R/P361R} mice treated by ICV administration of AAV9-CAG-hASAH1 at birth was comparable to wild type animals over the study period as assessed by using the four limb-hanging test, whereas the strength of untreated mutant mice did not increase over time (n=9 WT+PBS, n=11 Asah^{P361R/P361R}+PBS and n=11 Asah^{P361R/P361R}+AAV-hASAH1 mice at injection).

In conclusion, it is herein shown that a rAAV vector carrying the hASAH1 ORF can widely correct the phenotype associated to acid ceramidase deficiency. Indeed, the above results show that such a vector, administered via the IV or CSF route, can greatly prolong their lifespan, prevent weight loss and organ damage, restore muscle strength and has a positive effect on blood cell composition. This last result is particularly surprising because it was not expected that a rAAV9 vector carrying the hASAH1 ORF could have an effect on blood cell composition. Previous attempts to correct hematologic defects involved either hematopoietic stem cell transplantation or ex vivo gene therapy using hematopoietic cells genetically modified with lentiviral vectors, but these approaches did not have a significant effect on the manifestations of ASAH1 deficiency in the nervous system. In literature, it was reported that gene therapy approach for Farber disease by injection of hASAH1 ORF via lentiviral vector at 3 days of life failed to counteract growth retardation and survival rate over 120 days of life. Therefore, the findings herein shown also highlight the advantages on the use of an rAAV approach compared to lentivirus to perform gene replacement therapy able to globally correct disease phenotype both at pre- (3 weeks of life) and post- (7 week of life) symptomatic stage. Here, is provided a therapeutic strategy that will be useful to address the multiple damages induced by acid ceramidase disorders, in particular those induced in FD.

1-33. (canceled)

34. A method of treating acid ceramidase deficiency comprising administering a recombinant AAV (rAAV) vector comprising an ASAH1 open reading frame (ORF) to a subject having an acid ceramidase deficiency.

35. The method according to claim 34, wherein the acid ceramidase deficiency is a disorder selected from Farber disease (FD) and spinal muscular atrophy associated with progressive myoclonic epilepsy (SMA-PME).

36. The method according to claim 34, wherein the rAAV vector comprises an AAV9, AAV9P1, AAV10, AAVpo1A1, clade F AAVHSC, AAVrh.39 or AAV-PHP.B capsid.

37. The method according to claim 34, wherein the ASAH1 ORF is operatively linked to a ubiquitous promoter.

38. The method according to claim 37, wherein the ubiquitous promoter is a CAG promoter or a PGK promoter.

39. The method according to claim 38, wherein said rAAV vector comprises an expression cassette comprising, in this order:
a CAG promoter;
an ASAH1 ORF; and
a polyadenylation signal sequence.

40. The method according to claim 38, wherein said rAAV vector comprises an expression cassette comprising, in this order:
a PGK promoter;
a modified intron 2/exon 3 sequence from the human β globin gene;
an ASAH1 ORF; and
a polyadenylation signal sequence.

41. A recombinant AAV (rAAV) vector comprising:
i) a capsid selected from the group consisting of AAV9, AAV9P1, AAV10, clade F AAVHSC, AAVrh.39, AAVpo1A1 and AAV-PHP.B capsids; and
ii) a genome comprising an expression cassette comprising, in this order:
a CAG or PGK promoter;
an ASAH1 ORF; and
a polyadenylation signal sequence.

42. The rAAV vector according to claim 41, comprising:
i) an AAV9 capsid; and
ii) a genome comprising an expression cassette comprising, in this order:
a CAG or PGK promoter;
an ASAH1 ORF; and
a polyadenylation signal sequence.

43. The rAAV vector according to claim 41, wherein the promoter is a CAG promoter.

44. The rAAV vector according to claim 43, wherein the nucleic acid sequence of the CAG promoter consists of the sequence shown in SEQ ID NO:9, or a functional variant thereof which is at least 99% identical to SEQ ID NO:9.

45. The rAAV vector according to claim 41, wherein the promoter is a PGK promoter.

46. The rAAV vector according to claim 45, wherein the nucleic acid sequence of the PGK promoter consists of the sequence shown in SEQ ID NO:3, or a functional variant thereof which is at least 99% identical to SEQ ID NO:3.

47. The rAAV vector according to claim 41, wherein the expression cassette comprises, in this order:
a PGK promoter;
a modified intron 2/exon 3 sequence from the human β globin gene;
an ASAH1 ORF; and
a polyadenylation signal sequence.

48. The rAAV vector according to claim 47, wherein the modified intron 2/exon 3 sequence from the human β globin gene consists of the sequence shown in SEQ ID NO:4, or is a functional variant thereof which is at least 99% identical to SEQ ID NO:4.

49. The rAAV vector according to claim 41, wherein the polyadenylation signal is the HBB polyadenylation signal having a nucleic acid sequence consisting of SEQ ID NO:2, or a functional variant thereof which is at least 99% identical to SEQ ID NO:2.

50. The rAAV vector according to claim 41, wherein the expression cassette is flanked by an AAV2 5′-ITR and an AAV2 3′-ITR.

51. The rAAV vector according to claim 41, wherein the genome is single-stranded.

52. A method of treating acid ceramidase deficiency comprising administering a rAAV vector according to claim 41 to a subject having an acid ceramidase deficiency.

53. The method according to claim 52, wherein the acid ceramidase deficiency is a disorder selected from Farber disease (FD) and spinal muscular atrophy associated with progressive myoclonic epilepsy (SMA-PME).

54. The method according to claim 53, wherein the method treats at least one neurological and/or peripheral manifestations of FD.

55. The method according to claim 54, wherein the method treats at least hematological manifestations of FD or treats neurological and hematological manifestations of FD.

56. The method according to claim 52, wherein the rAAV vector is administered intravascularly.
